(19) United States
(12) Patent Application Publication
(10) Pub. No.: US 2025/0228800 A1
(43) Pub. Date: Jul. 17, 2025

(54) Treatment of nasal cavity inflammation and mucus hypersecretion.

(71) Applicant: Edward T. Wei, Berkeley, CA (US)

(72) Inventor: Edward T. Wei, Berkeley, CA (US)

(21) Appl. No.: 18/831,174

(22) Filed: Aug. 29, 2024

Related U.S. Application Data
(63) Continuation-in-part of application No. 18/445,746, filed on Jan. 12, 2024, now abandoned.
(63) Continuation-in-part of application No. 18/445,766, filed on Jan. 25, 2024, now abandoned.
(63) Continuation-in-part of application No. 18/445,901, filed on Mar. 14, 2024.

(51) Int. Cl. A61K 31/16; A61M 3/02

(52) U.S. Cl. CPC A61K 31/16; A61M 3/0262; A61M 2210/0618

(57) ABSTRACT

In screening cooling agents for their actions in the oral cavity, the molecule FEMA 4557, registered for use in chewing gum and hard candy, produced a long-lasting, stinging, and painful cold, like ice chips on the throat. To further study its properties, FEMA 4557 was formulated as a topical gel and shown to relieve skin discomfort without causing any skin injury or irritation. FEMA 4557 relieved inflammation from flea bites and a bumble bee sting and relieved the inflammation of dermatological disorders such as skin allergy, urticaria, and thermal injury. The duration of the anti-inflammatory action was for several hours after a single application. The pharmacological potency of FEMA 4557 did not correlate to the EC₅₀ potency at the TRPM8 receptor. Surprisingly, when FEMA 4557 was delivered to the inflamed nasal mucosa with a compressor-nebulizer, it relieved nasal stuffiness and inhibited phlegm secretion in subjects with rhinitis, as measured by patient-outcome responses and an objective gravimetric method, respectively. FEMA 4557 was also effective for treating rhinitis when administered in nose drops or in a rinse. The effective therapeutic amount of FEMA 4557 was about 1 to 5 mg per retained dose. The conclusion is that FEMA 4557 is a potent non-irritating suppressor of acute inflammation in the nasal cavity and sinuses, a property not previously described for other topical cooling agents. Thus, FEMA 4557 and related analogs may have therapeutic utility for treating nasal inflammation and mucus hypersecretion.

This application is a continuation-in-part of Ser. No. 18/445,746 filed Jan. 12, 2024, Ser. No. 18/445,766 filed Jan. 25, 2024, and Ser. No. 18/445,901 filed Mar. 14, 2024.

### BACKGROUND OF THE INVENTION

Field of Invention One aspect of the present invention is a method of treatment of rhinitis and rhinosinusitis, wherein the treatment reduces nasal stuffiness, nasal congestion, and phlegm secretion. About 15% of the general population suffers from rhinitis. In this application, the preferred embodiment, FEMA 4557, is an example of a molecule, topically applied to the nasal mucosa, that can be used to treat rhinitis. On the skin, FEMA 4557 reduced pain, itch, swelling and promoted rapid wound healing. When tested on the nasal mucosa, treatment reduces nasal stuffiness, nasal congestion, and mucus hypersecretion. A typical example of nasal mucus hypersecretion is the excess secretions produced by viral infections. Another condition of hypersecretion is rhinosinusitis caused by inhaled allergens or noxious substances. Reducing mucus hypersecretion in rhinosinusitis relieves nasal stuffiness and allows more comfortable breathing.

Description of the Related Art Air passes into and out of the lungs via the nasal cavity. The average nasal cavity volume in adults is around 15.5 cc for men and 13.5 ccs for women. The surface area of the nasal cavity is about 150 to 200 cm² for adults (about the size of one surface of the hand). The nasal cavity is lined with specialized epithelia, nerves, and mucosa that warm and humidify the inhaled air. The paranasal sinuses are air-filled cavities within the bones of the face that connect to the nasal cavity via apertures called ostia. The ostiomeatal complex (OMC) (FIG. 10,11) is an anatomical region in the nasal passages and sinuses in the center of the head. It consist of a series of small openings (ostia) that connect the frontal, maxillary, and anterior ethmoid sinuses to the middle meatus of the nose. Also, part of the OMC is the sphenoidal sinus and posterior ethmoidal cells that drain into the superior meatus. Accumulation of fluids and mucus in the nasal cavity and sinuses impedes breathing and is removed by either blowing the nose or voiding via the nasopharynx and swallowing.

Mucosal secretions of the sino-nasal cavity come from serous, mucinous, and sero-mucinous glands. Serous fluids are transparent and watery and contain electrolytes and proteins, such as amylase and lysozyme. By contrast, mucus is thick, gel-like, viscous, and contains mucins (large glycoproteins). The mucus is ˜95% water, but when dried, it forms “snot” and crusts in the nasal cavity. Dried mucus can be as hard as a rock. Mucus can be a nutrient source for infections and, together with inflammatory exudates, form “phlegm.” For a healthy and clean nasal cavity, a subject must expel excess phlegm by blowing or swallowing. Otherwise, the phlegm obstructs and, when desiccated, impacts the epithelia and becomes a source of pathophysiology. Mucus hypersecreted into the nasal cavity and sinuses will accumulate and impede breathing unless removed. It has been recognized for some time that excess mucus is not healthy for the airways, but effective methods for reducing mucus in airways are limited and have not been well characterized.

Inflammation of the nasal cavity and sinuses are common conditions called rhinitis and sinusitis. The surfaces of the nasal cavity and the sinuses are contiguous, so drainage of inflammatory exudates follows a common pathway. Chronic rhinosinusitis (CRS) is inflammation of the sinonasal passages lasting 8 to 12+ weeks. The symptoms and signs are nasal stuffiness, congestion or blockage, nasal discharge that may be thick, purulent, and discolored, facial pain, pressure or fullness, and changes in the sense of smell (hyposmia or anosmia). Radiological imaging, such as computer tomography (CT) scans, helps diagnose sites of opacification and impaired sinus drainage.

A prominent complaint of patients with rhinitis and sinusitis is “stuffiness,” or the subjective feeling of impaired airflow, loss of patency, and uncomfortable breathing. For example, one can lie down to sleep with the common cold and discover that breathing is difficult. Mouth breathing is not a satisfactory alternative and leads to disturbed sleep. The term “congestion” is often used interchangeably with stuffiness. However, congestion more precisely refers to evidence of physical blockage or obstruction of the nasal passage due to swelling or excess mucus. This type of evidence is difficult to obtain in the clinic. The sense of impaired breathing or stuffiness affects sleep and patients' quality of life with rhinitis and rhinosinusitis. Patients frequently focus on this complaint as the most undesirable and annoying source of discomfort.

Inflammation is the reaction of vascularized living tissues to injury. The underlying triggers and mechanisms of rhinitis vary. Allergic rhinitis is an immune system response to allergens. Inflammation in the nasal passages occurs when the mucosae overreact to allergens. Common allergens include pollen from trees, grasses, weeds, dust mites, pet dander, mold spores, and specific food allergens. Allergic rhinitis tends to occur seasonally but may also be perennial. Non-allergic rhinitis has triggers such as viral or bacterial infections, inhaled irritants such as odors, perfumes, and smoke, changes in temperature or humidity, hormonal changes, or occupational exposure to chemicals, dust, or fumes. It is estimated that 60 million and 30 million individuals in the USA have allergic and non-allergic rhinitis, respectively. The number of patients with CRS is not known with precision because diagnostic criteria vary. The diagnostic criteria for CRS are rigorous and if drug treatment fails, the next step is frequently surgery.

There are several drug treatments for rhinitis. For example, seasonal rhinitis is treated with topical or systemic antihistamines such as azelastine and fexofenadine (Allegra®) and by corticosteroids such as mometasone, budesonide, or fluticasone. However, patient adherence to these medications is low (<30%). A muscarinic antagonist, ipratropium, is also used for drying the nasal mucosa in rhinosinusitis. Decongestants (sympathomimetic amines) are less favored as drugs because of the risks of rebound hyperemia. Vaporub, composed of camphor, menthol, and eucalyptus oil has been available for 100+ years, but it is pungent and can irritate. Other adjuncts of treatment are nasal irrigation and antibiotics. Newer drugs for CRS have been introduced based on monoclonal antibodies to scavenge interleukins (IL-4, IL-5, and IL-13). Such drugs, e.g. dupilumab or mepolizumab act on the immune system. A new strategy is proposed in this application.

Surgery is an alternative when drug (or “medical”) treatments fail to control symptoms, Endoscopic sinus surgery is a procedure to remove obstructions, inflamed tissues, and nasal polyps. Removal is expected to facilitate clearance and drainage of nasal passages. It is usually performed under general anesthesia using an endoscope, a thin, flexible tube with a camera and light attached to its end. The endoscope allows the surgeon to visualize the inside of the nose and sinuses without making large incisions on the face. Typical structures removed are parts of the uncinate process and lower turbinates. More recently, rhinologists have been using cryosurgical and electrolytic procedures to remove the nerves of the nasal cavity to treat recalcitrant rhinitis. As the patient base for rhinitis and sinusitis is large, there is a need for further innovation.

### BRIEF SUMMARY OF THE INVENTION

One aspect of the present invention is a method of treating rhinitis and mucus hypersecretion in the nasal cavity in a subject in need of treatment. The embodiment is a TRPM8 agonist called FEMA 4557, formulated in a liquid suspension or as a solution and topically administered onto the surface of the subject's nasal cavity in a nebulized spray, nose drops, or as a nasal rinse. The TRPM8 agonist, FEMA 4557, has shown significant effectiveness in reducing mucous secretions, as evidenced by the objective measurement of the treated subject's nasal blowouts or the subject's self-reported description of nasal relief. Thus, one surprising discovery was that the topical cooling agent inhibited phlegm production. Beneficial effects were observed within 15 min of delivery of the preferred embodiment and lasted for about six hours after a single delivery. Multiple doses appear to confer permanent benefits for subjects with rhinitis and mucus hypersecretion.

In the design strategy for a new drug to treat rhinitis the specifications for a topical medication include descriptions of a) the target drug receptor and its anatomic location, b) a method of drug formulation, c) a method of drug delivery, d) a measurement of treatment outcome, and e) a possible mechanism of drug action.

The drug target is the TRPM8 receptor located on nerve fibers of the nasal cavity, a crucial location for the treatment of rhinitis and mucus hypersecretion. The active ingredient is classified as a TRPM8 agonist. FIG. 10 shows a photomicrograph of the TRPM8 target in a nerve fiber on the mouse nasal mucosa. These TRPM8 receptors may be densely congregated in the ostiomeatal complex (OMC) (FIG. 11), a central, convergent point of the nasal passages and sinuses, which makes it an ideal target for the treatment.

The TRPM8 agonist is formulated as a liquid solution or as a liquid suspension for even distribution onto the nasal surface. A standard carrier solution is isotonic saline. The delivered volume and concentration of the active ingredient determine the dose. The volume per nose may vary from 0.5 to 1 mL per dose for a nebulized spray or 5 to 15 mL per dose for nose drops or a rinsing solution. The concentration of an active ingredient such as FEMA 4557 may vary from 5 to 15 mg/mL for the spray or nose drops, and from 0.2 to 2 mg/mL for the rinsing solution. These methods of delivery are straightforward and ensures treatment can be administered with minimal disruption to the patient's daily routine.

One method of precise and accurate drug delivery to the target receptor is by use of an electronically controlled nebulizer (FIG. 13, e.g., NasoNeb device). This device delivers a specific volume of the drug per nostril, ensuring accurate dosing. Under standardized compression, the jet going past the nasal valve is retained in the middle meatus and preferentially delivered to the OMC (FIG. 11,12). The particle sizes of the aerosols is carefully controlled, to facilitate the drug reaching the target receptor. A second method is to use saline irrigation bottles (FIG. 13, 14, e.g., NeilMed Sinus Rinse and Water Pulse). The terms “nasal irrigation,” “nasal lavage,” “sinus wash,” and “nasal wash” are synonymous and refer to saline introduced into the nasal and sinus cavities for cleansing or medicating. These procedures are now familiar and common. The solution plume used in the rinse is generated from containers by manual compression and the volume can range from 2 to 20 mL per activation. The NasoNeb nebulizer, nose drops, and rinse method are better than aerosolized sprays because drug entry into the lungs is minimized (FIG. 12). The saline irrigation system of this discovery is defined as comprising a) a container of 5 to 300 mL in volume b) ˜isotonic saline and c) a medication in the saline that is delivered to the nasal cavity surface by a plume of water entering one nostril and exiting via the other nostril. The medication is delivered en passage in the nasal cavity.

The outcome measurement in the treated subject can be a visual analog index of the sense of nasal patency or openness in breathing. An objective sign of response to therapy is the gravimetric output of nasal exudates measured with a microbalance.

The choice of the best TRPM8 agonist for the practice of this invention is from an arsenal of molecules known to the art or synthesized de novo by the applicant. Examples of optimized candidates for delivery and therapy are presented. The first selection of FEMA 4557 (Formula 1) is based on its formulation properties and ease of delivery. FEMA 4557 has a total mass of 199.12 Daltons which is lower than most other cooling agents which ≥200 Daltons. This lower mass of FEMA 4557 improves its dispersion in liquids and penetration in tissue fluids, enhancing its effectiveness in the treatment. In preliminary studies described here, FEMA 4557 exerts an anti-inflammatory activity at topical concentrations on the skin of ≥5% wt/vol and a total dose of several mg. FEMA 4557 also stops pain and itch and exhibits unusual healing properties on injured tissues. Surprisingly, when applied to the inflamed nasal membranes in a rinsing solution, FEMA 4557 0.1% wt/vol stops the stuffiness of rhinitis and inhibits the secretion of phlegm. Thus, FEMA 4557 is a prototype of a new class of drugs for the treatment of rhinitis.

The principal complaint of patients with rhinitis and sinusitis is “stuffiness,” or the subjective feeling of impaired airflow, loss of patency, and uncomfortable breathing. The mechanisms underlying the sensation of stuffiness have undergone revolutionary change in the past 20 years. David Julius and Ardem Patapoutian (Nobel Laureates of 2021) discovered that TRP (transient receptor potential) and other (piezo) ion channels on nerve endings underlie the sensations of hot and cool and pressure (mechanoreceptors). Previously, “congestion” and “obstruction” were often used interchangeably with stuffiness. However, the evidence of stuffiness caused by reduced nasal cavity volume or airflow resistance was lacking. Instead, the modern view is that stuffiness is caused by disturbed thermo- and mechano-sensations on the nasal mucosal surfaces. The respiratory epithelium of the nasal cavity is one cell layer thick, and the mucosa is densely innervated. Like the ocular surface, the nasal sensitivity to cooling agents is ≤1 mg/mL, compared to the pharynx, which is ≥5 mg/mL. The nasal cavity surface, like the eye, is highly sensitive to the presence of foreign bodies. Thus, dried mucus (phlegm, snot, crusting) easily disturbs breathing and causes nasal discomfort. Patients complain of stuffiness because of disruption in thermal and mechano sensations on the nasal surfaces. These noxious sensations are overcome by the coolness produced by a TRPM8 agonist. Surprisingly, the preferred embodiments also inhibited nasal secretion of phlegm, the principal source of mechano-obstruction.

Examples are described herein wherein a) a cooling agent, nebulized with an electronically controlled compressor, relieves the symptoms of rhinosinusitis and inhibited phlegm secretion, b) a cooling agent in nose drops administered to a subject with allergic rhinitis and post-nasal drip, reduced cough and enabled better sleep, and c) a cooling agent dissolved in saline solution and administered with nasal irrigation bottle relieved rhinitis and stuffiness. The beneficial effects of topical medication, relief of nasal stuffiness and inhibited nasal secretion of phlegm, appear within 5-10 min after administration and may last for about six hours after a single delivery. Surprisingly, multiple doses appear to confer permanent benefits.

In an example of the practice of this invention, a nebulizer with an electronically controlled compressor (NasoNeb) was used to deliver the cooling agent FEMA 4557 (liquid suspension) to the ostiomeatal complex (OMC). FEMA 4557, a food additive used in confectionery, was formulated at 0.5 to 1.5% wt/vol solution (5 to 15 mg/mL) and delivered at a volume of ˜0.5 mL/nostril over 3 to 5 sec with the designed nebulizer. A dosage schedule of two to three times per day for three to five days lowered phlegm output, objectively measured with a microbalance. The subject felt that his nasal cavity was clear. He slept well and did not need to blow his nose. Other examples using nose drops and rinsing solutions are described.

In summary, the drug target is the TRPM8 receptor on the nerve fibers of the nasal cavity. The active ingredient is a TRPM8 agonist formulated for drug delivery via a nebulizer, nose drops, or rinse. The quantification of response is the output of nasal exudates measured with a top-loading microbalance or subject-reported outcome. The choice of the best TRPM8 agonist is based on experiment. Examples of optimized candidates for delivery and therapy in the practice of this invention are presented.

### BRIEF DESCRIPTION OF THE SEVERAL VIEWS OF THE DRAWINGS

FIG. 1. Comparison of cooling intensity of substances tested in the oral cavity at 100 ppm in 5% sucrose. Cooling intensity was self-rated on a scale of 0 to 9 units. The data shown in this Figure is from Sonya Johnson et al., 2017. The rank order of potency of WS-5>WS-3˜I-menthol>WS-12>WS23 agrees with other compilations published by Leffingwell (2014) and by Erman (2007).

FIG. 2. Median Effective Dose (EC₅₀) of various cooling agents tested on TRPM8 in an in vitro assay. The consensus is that icilin and WS-12 are the more potent agonists, followed by WS-5>WS-3˜I-menthol. The acyclic amides WS-23 and FEMA 4557 are significantly less active than I-menthol. The applicant provided the test substances used in the publications by Mckemy et al., Bodding et al., and Sherkheli et al. Further experiments by the applicant confirmed the weak activity of FEMA 4557 on TRPM8 (10% the activity of I-menthol) and the absence of activity on TRPA1.

FIG. 3. Bioassay of FEMA 4557 (labeled as BUDA) in TRPM8 and TRPA1. Data from the laboratory of Prof. Seok-Yong Lee's at Duke University. The EC₅₀ of FEMA 4557 was estimated at 0.41 mM or about 30× less potent than I-menthol. FEMA 4557 was not active in cells transfected with TRPA1, although a positive TRPA1 control AITC (allyl isothiocyanate) was active.

FIG. 4. Cooling agents, prepared as 8% gel, were tested topically on the philtrum skin assay. The duration of cooling was recorded (hr on y-axis). The startling activity of FEMA 4557, cooling for ˜3 hr., was unexpected and surprising. FEMA 3784 is also known as Cool 10 or menthoxypropanediol.

FIG. 5. Dose-response relationship of FEMA 4557 to cooling on the philtrum skin. FEMA 4557 was dissolved in a polymer gel (Carbopol® 980) and applied to the philtrum skin with a cotton-tipped applicator. Cooling intensity area-under-curve (AUC) duration in hours was recorded on a standardized philtrum skin assay.

FIG. 6. The skin's inflammatory response to flea bites was treated with topical FEMA 4557. Half an hour after the bite (left panel), the skin is swollen, and intensely itchy. After application of FEMA 4557, 32% solution, swelling and redness, is reduced (middle panel, 12 hr after bites). Surprisingly, redness, swelling, and itch significantly diminishes with a single application of FEMA 4557 and the skin heals quickly (right panel, 24 hr). Thus, a single application of FEMA 4557 is sufficient to counteract inflammation.

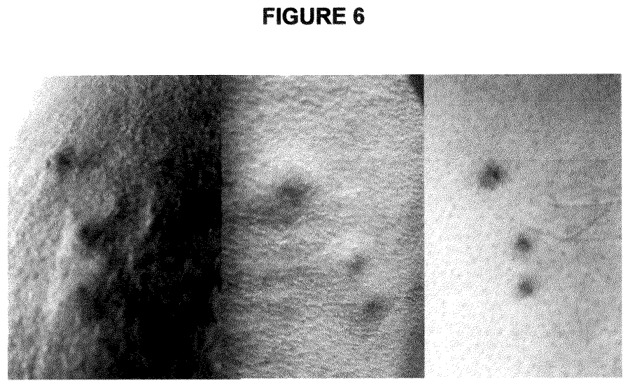

FIG. 7. Inhibition of pain and swelling from a bumble bee sting with FEMA 4557. The subject's distal phalanx of the middle finger was stung by a bumble bee when he tried to grab the bee with tissue paper. The volume of the distal phalanx increased from 7.0 g to 9.0 g within 30 min, as measured by the fluid displacement method over a top-loading balance. The swelling was accompanied by redness and pain. The left panel shows the normal middle finger of the contralateral hand. Half an hour after the bite (middle panel), the distal phalanx is swollen, and painful. The application of FEMA 4557, 32% solution, onto the finger is shown in the right panel. Surprisingly, the pain in the middle finger was converted to an intense itch within several minutes. This conversion of pain to itch is unexpected, and more tolerable to the subject. The itching lasted several hours.

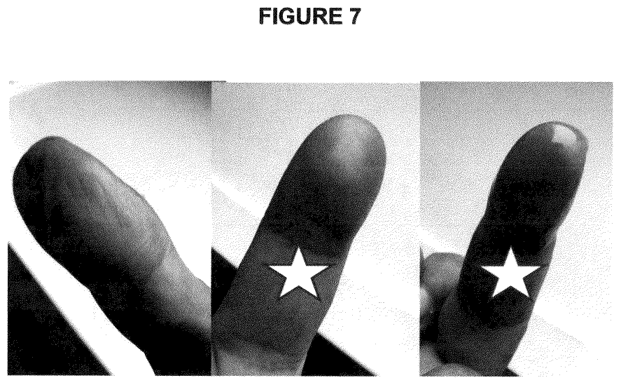

FIG. 8. A male subject burned his hand with a 4-jet butane fueled cigar lighter. The burn site (white circled area in Figure) rapidly became swollen. A 20% solution of FEMA 4557 was applied with a cotton-tipped applicator to the burn site 15 min after the burn. Surprisingly the pain together with redness and swelling rapidly diminished and was not noticeable after 5 hr. FEMA 4557 was only applied once. The wound site formed an eschar which resolved within three weeks.

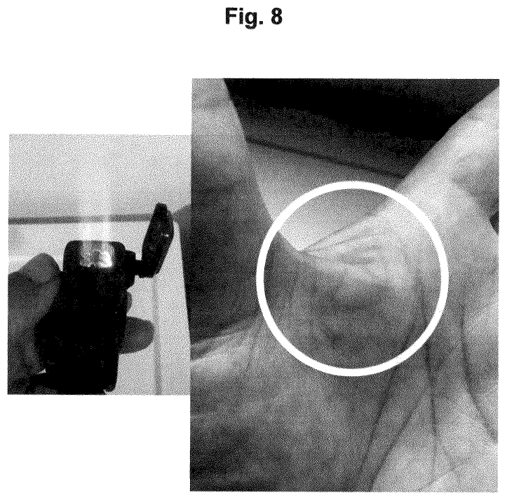

FIG. 9. A 53-year-old female subject developed recurrent episodes of urticaria six months after receiving a Covid-19 vaccination. Each episode of severe itching lasted ˜2 weeks, and there was 10+ episodes. The rash occurred mainly on the neck and trunk. Inspection of the nape showed evidence of scratching (lighter shade skin where the skin was abraded, white circle). Applying a 16% wt/vol FEMA 4557 gel controlled the itch, allowing the subject to rest and sleep.

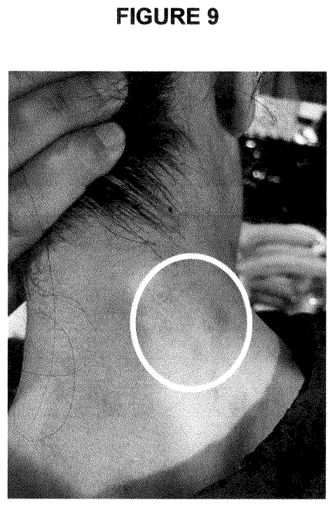

FIG. 10. Photomicrograph of TRPM8 receptor on mouse nasal mucosa nerve fiber [Wei. Medical Hypotheses 142 (2020) 109747]. The histology is described in Yang et al., 2017. The TRPM8 fiber was identified by green fluorescent protein methods in transgenic mice (Trpm8+/EGFP). The TRPM8 receptor on the nasal mucosa is the drug target.

FIG. 11. The ostiomeatal complex (OMC) is the intended anatomic site for drug delivery via an electronic nebulizer, nose drops, or a rinsing solution. The lateral walls of the OMC contain the ostia of the sinuses, three in the middle meatus and two in the superior meatus. Delivery of the TRPM8 agonist is intended to relieve stuffiness (via cooling sensations) and facilitate drainage of the sinuses via the ostia and the nasal cavity via the nares and nasopharynx. The anatomic complexity of the OMC is apparent in this illustration from Gray's Anatomy. A positive pressure rinse is one optimal drug delivery method to the OMC.

FIG. 12. Characteristics of intranasal drug delivery unit for the preferred embodiments using a liquid plume. On the left, the jet of the NasoNeb® nebulizer illustrates the strength of electronic activation. Over 99% of the particles generated from the NasoNeb® canister are >10μ in diameter and hence do not enter the lungs. Manual sprayers (middle two plumes) are more variable and generate aerosol particles that may enter the lower airways. On the far right, a squeeze bottle (NeilMed, Inc.) generates a plume for rinsing the nasal cavity. Nose drops and a rinse from a squeeze bottle create liquid droplets that are too large to enter the lower airways.

FIG. 13. The NasoNeb® nebulizer, used in the present studies, wherein >99% of the particles generated from the canister are >10μ in diameter and thus do not enter the lungs. A bottle containing the test solution is used to fill the canister. The jet from the electronically controlled compressor is more powerful than manual sprayers or irrigators. The liquid suspension of the preferred embodiments delivered over three to five seconds per nostril is sufficient for efficacious treatment.

FIG. 14. Examples of devices for irrigating the nasal cavity with saline. On the left (WaterPulse) is a device with a 300 mL reservoir bottle. The white button on the bottom of the bottle can be used to control outflow. On the right is a device (NeilMed, Inc.) with a 240 mL reservoir bottle. The plume of liquid is activated by manual compression of the plastic bottle. The NeilMed Sinus Rinse device was described by Mehta (U.S. Pat. No. 6,520,384) and used in the present discovery.

FIG. 15. Quantifying inflammatory response of nasal mucosa. The secretions and exudates of the nasal cavity were measured. Subjects were instructed to blow into a pre-weighed paper tissue and record the weight of the tissue after blowing. The tissue was then inspected and marked as clear, with streaks of exudates, or with dense gel. Surprisingly, the nasal blowouts fell into three distinct weight categories that were easy to recognize. The blowout weights thus provided an objective index of nasal cavity inflammation.

FIG. 16. Inhibition of nasal secretions by topical administration of FEMA 4557, dissolved at 15 mg/mL in an ethanol, polysorbate 80, propanediol mixture, in a subject with rhinitis. Delivery into the nasal cavity was with a Nasoneb® apparatus. The test substances were rinsed into the nasal cavity for 5 sec at t=0, 2, and 5 hr. The data points were fitted by linear regression (GraphPad Prism) and the 10-hr secretion measured as (avg.±s.e.m). The results were vehicle 3.16±0.10 g, and FEMA 4557 1.86±0.07 g, with n=6, 5 observations per group, respectively. The nasal secretion-10 hr unit for FEMA 4557 was significantly lower (P<0.01) than vehicle when analyzed by the Kruskal-Wallis test for non-parametric data.

FIG. 17. Inhibition of nasal secretions by topical administration of FEMA 4557, dissolved at 15 mg/mL in an ethanol, polysorbate 80, propanediol mixture, in a subject with rhinitis. Delivery into the nasal cavity was with a Nasoneb® apparatus. The test substances were rinsed into the nasal cavity for 5 sec at t=0, 2, and 5 hr. The data points are for individual “blow-outs” from the nose, measured with a tissue paper and balance. The average±s.e.m. for blowouts were: vehicle 0.38±0.03 g and FEMA 4557 0.21±0.03 g, with n=61, and n=56 observations per group, respectively. The blowout units were significantly different (P≤0.0001) when analyzed by the Kruskal-Wallis test for non-parametric data.

### DETAILED DESCRIPTION OF THE INVENTION

One aspect of the present invention is a method of treatment of rhinitis and rhinosinusitis, wherein the treatment reduces stuffiness, nasal congestion, and phlegm secretion, in a subject in need of treatment thereof. An embodiment is whereby a p-menthane carboxamide formulated for nebulization in a liquid suspension or as a solution is provided and then topically administering the p-menthane carboxamide formulation to the ostiomeatal complex of the subject's nasal cavity with an electronically controlled nebulizer. The topical medication will relieve nasal stuffiness and inhibit nasal secretion of phlegm. Beneficial effects are observed for about six hours after a single delivery, and multiple doses appear to confer permanent benefits. Treating a patient with severe rhinitis or rhinosinusitis relieves symptoms and enables the patient to have restful sleep.

Cooling to treat nasal stuffiness and congestion is intuitively comprehensible. We know that breathing cool air reduces the sense of stuffiness. Monoterpenoids such as menthol, camphor, and eucalyptol oil are traditional chemicals used to “clear sinuses.” For example, Vicks VapoRub appeared in 1911, and the active ingredients are camphor (4.8%), menthol (2.6%), and eucalyptus oil (1.2%). The side effects of monoterpenoid vapors, e.g., pungency, eye irritation, rhinorrhea, and the short duration of action, limit their use. As described here, an electronic nebulizer delivers the preferred embodiments, which act within minutes and last for hours. Nasal stuffiness is gone, and the subject can use the spray as needed. The drug effect is “cool esthesia” because it relieves the dysesthesia of rhinitis.

Surprisingly, the preferred embodiments also inhibited nasal secretion of phlegm. When secreted by the mucous gland, the mucus is a transparent white protein, but when mixed with other secretions and inflammatory exudates, it becomes nasal phlegm. This phlegm is the principal pathophysiological element in rhinitis. Nasal phlegm, also referred to herein as “gunk,” is the yellowish-green slimy substance copiously secreted by the glands of the nasal mucosa when inflamed. The principal element of gunk is a mucopolysaccharide called MUC5B, which absorbs water from the environment and swells to form a gel. When the gel dries, it becomes a crusty snot and can be as hard as shoe leather. Removal of gunk is necessary for a healthy and open nasal passage. Otherwise, it will coat and obstruct airflow and impede thermal exchange in the nasal cavity. The opaque materials in an X-ray that shows blocking of the nasal passages and sinuses and defining the state of severe chronic rhinitis is desiccated gunk.

TRPM8 Target and Anatomic Location. In the past 20+ years, scientists have realized that cooling is a molecular event conveyed by dedicated nerve fibers containing TRPM8 (an integral membrane protein receptor). These nerve fibers fire when tissue temperatures drop below 25° C., stimulated by negative heat flux or chemical agents. Moreover, cooling is modality-specific, and its anatomic circuitry has its own set of cables. TRPM8-mediated cooling is the physiological basis for “cool esthesia.” TRPM8 agonists replicate a negative heat flux sensation from the nasal membranes. Surprisingly, when these molecules are rinsed onto the nasal cavity surfaces, gunk production declines. The experiments for finding an effective molecule and the appropriate delivery parameters are described here.

The anatomic target for sustained relief of nasal stuffiness and exudate secretions in the practice of the present invention is the TRPM8 receptors on nerve fibers of the ostiomeatal complex (OMC). The OMC is a central region in the nasal passages and sinuses. It consists of a series of small openings (ostia) that connect the frontal, maxillary, and anterior ethmoid sinuses to the middle meatus of the nose. The OMC is posterior to the nasal valve (the narrowest area of the nasal cavity with an area of about 0.7 cm², about 1.3 cm behind the nares). This surface is not easily accessible with conventional sprays or nose drops. An electronically controlled nebulizer or a rinsing solution for drug delivery are the preferred methods of topical administration of the preferred embodiment. Successful treatment of patients with rhinosinusitis means relieving symptoms of nasal stuffiness and diminishing the sign of phlegm secretion. Both indices define therapeutic success.

Understanding nasal secretions is essential to treating rhinitis. When secreted by the mucous gland, mucus is a transparent white protein, but when mixed with other secretions and inflammatory exudates, it becomes nasal phlegm. This phlegm is the principal pathophysiological element in rhinitis. Nasal phlegm is the yellowish-green slimy substance copiously secreted by the glands of the nasal mucosa when inflamed. The principal element of phlegm is a mucopolysaccharide called MUC5B, which absorbs water from its environment and swells to form a gel. When the gel dries, it becomes a crusty snot and can be as hard as shoe leather. Removal of excess phlegm is necessary for a healthy and open nasal passage. Otherwise, it will coat and obstruct airflow, irritate like a foreign body, impede thermal exchange in the nasal cavity, and interfere with mucociliary clearance. The opaque materials in an X-ray that show blocking of the nasal passages and sinuses and defining the state of severe chronic rhinitis is desiccated phlegm.

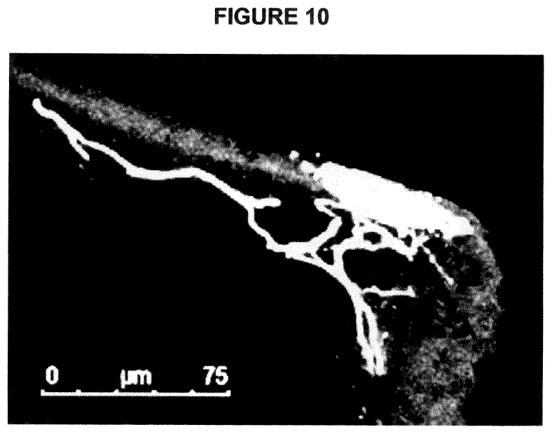

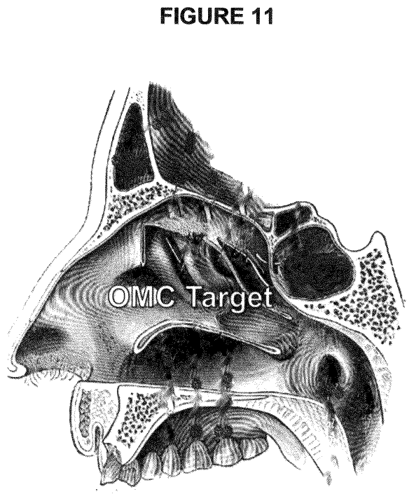

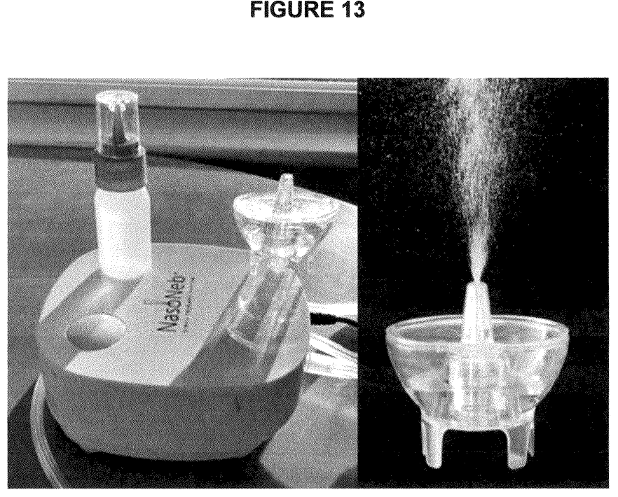

In summary, the drug target is the TRPM8 receptor on nerve fibers of the OMC (FIG. 10,11). The active ingredient is a TRPM8 agonist. FIG. 11 shows a photomicrograph of the TRPM8 target in a nerve fiber on the mouse nasal mucosa. The method of drug delivery is an electronically controlled nebulizer (FIG. 13). The choice of the best TRPM8 agonist is from an arsenal of molecules known to the art or synthesized de novo by the applicant. The quantification of response is the subjective sensations of nasal patency or openness in breathing, and the objective sign of therapy is the output of nasal exudates measured with a microbalance. Examples of optimized candidates for delivery and therapy are presented.

Formulation of Active Ingredient. Formulations for nebulization with NasoNeb® used a standard recipe of (Table 6): 1% of the active ingredient (1 to 10 mg/ml), 2% Tween 80 (Polysorbate 80), 1 to 5% ethanol, and made up to 100% with water. The suspension is mixed and centrifuged in a FlackTek, Inc., Speedmixer for at least 10 min at 3000 rpm. The Speedmixer spins the sample in 3 dimensions. The result is a white homogeneous liquid suspension with some of the solids now dissolved. For dosing, ˜2 to 3 mL is loaded into the canister of the nebulizer. The nebulizer nozzle tip is placed in the nostril and activated by pressing a button for 3 to 5 sec using a clock timer. The delivered volume was remarkably consistent at for a 3-see and 5-see activation resulted in 0.62±0.1 mL and 0.73±0.1 mL delivered per nostril, respectively. The nasal cavity is relatively tolerant to exogenous agents with irritant properties, as many rinses are used for rhinitis and sinusitis.

Drug Delivery Devices. Irrigation of the nasal cavity with saline has become an accepted method of hygiene for the treatment of rhinitis. Many devices are available for cleansing, ranging from a simple moisturizing nasal spray (˜0.5 mL per activation) to a plastic bottle with a liquid capacity of ˜250 to 300 mL and delivering 5-12 ml per activation. Three methods of such delivery, an electronically-controlled nebulizer, nose drops, and a rinse, have the advantage of avoiding spray droplets of a size that might enter the lungs. Entry of a chemical entity into the lungs entail more safety tests and expenses. These three methods, described in greater detail below, were used to deliver a TRPM8 cooling agonist to the surface of the nasal cavity.

A unit for focused delivery of small volumes (≤1 mL per nostril) to the ostiomeatal complex is available. It can be called a nebulizer or sprayer. A nebulizer and a sprayer disperse liquids into droplets, but they operate differently. A nebulizer is conventionally used to deliver medication as mist inhaled into the lungs. Such formulations and methods are common for asthma, chronic obstructive pulmonary disease, or other lower respiratory disorders. Nebulized aerosols enter via the oral cavity. A sprayer is a general term for any device dispensing aerosols over a surface. Hand-held sprayers are often used medically for delivering liquids into the nasal cavity, with the tip inserted into the nostril. The sprayer uses manual air pressure to force the liquid out of the nozzle, breaking it into airborne droplets. The aerosol disperses over the target area. Usually, the particle size distribution, dispersion pressure, and volume of a hand-held sprayer are not exact because of user differences. Hand-held sprayers are used to deliver intranasal steroids, antihistamines, and decongestants for the treatment of rhinitis. These drugs are effective for allergic rhinitis but not viral rhinitis. The steroids have some efficacy for chronic rhinosinusitis but are of marginal value in non-allergic rhinitis.

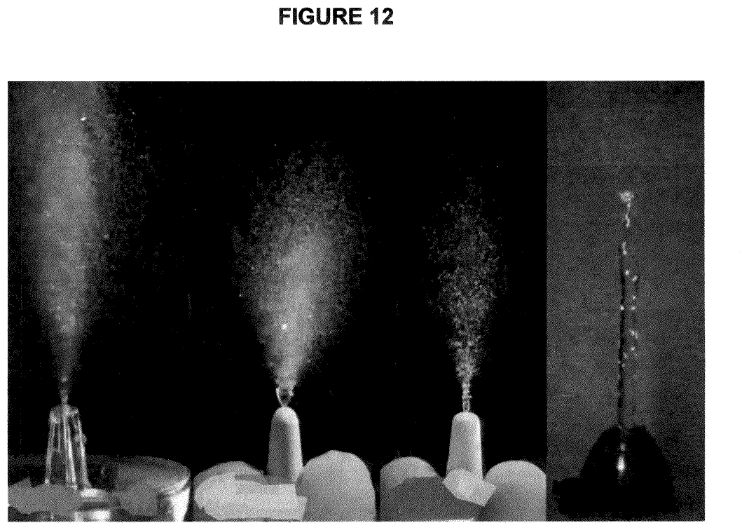

Flickinger (U.S. Pat. Nos. 5,906,198 and 8,892,544) described a electronic nebulizer optimized for selectively delivering particles into the nasal and paranasal cavities. This system utilizes an electronically controlled compressor to generate large liquid particles. Over 99.9% of the particles are >10μ. These particles get trapped in nasal and sinus cavities and do not enter the lungs. This system is available as NasoNeb® Nasal Nebulizers. The nebulizer used here is Model 7070NasoNeb® Sinus Therapy System. The use of this device helped enable the discovery of this application. The nasal and sinus cavities capture virtually all particles delivered via the NasoNeb Nebulizer (FIG. 13). Under standardized compression, the plume going past the nasal valve is retained in the middle meatus and delivered to the OMC with maximum effect (FIG. 12). The areas reached by the nebulized large particles include the frontal recess/sinus, spheno-ethmoid recess, ethmoid cavity, sphenoid and maxillary sinuses, turbinates, the middle meatus, and olfactory cleft. Conventional manual sprayers and Metered Dose Inhalers (MDIs) produce smaller particles that mainly reach the anterior third of the inferior and middle turbinates. These devices do not have a positive pressure generator and lose momentum after exiting the nozzle. The pump creates positive pressure and an air column for the nebulizer to propel particles past the nasal valve and deeper into the nasal cavity. The particle size distribution with the nebulizer is such that particles are less likely to reach the pharynx and to cause adverse effects such as laryngeal irritation or voice alterations.

The precise electronic control of the compressor in the nebulizer allows accurate calculation of the administered dose. Experimentally, the nebulizer delivers 0.35 to 0.75 mL into the nasal cavity in a 3 to 5-second activation. This control of the rate and volume of liquid delivery permits quantitative comparisons of the specificity and selectivity of the preferred embodiments. The parameters of the nebulizer pump may be adjusted for various delivery times, e.g., 5 to 10 sec, while preserving the desired particle size distribution. A lightweight hand-held device weighing ˜300 g, with a reservoir canister of 5 mL and a battery power source, may be optimal for individual use.

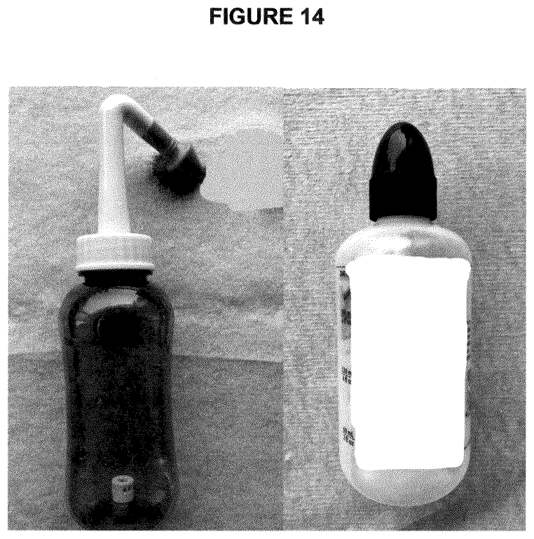

Saline irrigation systems, bottles designed for holding saline solutions to irrigate the nasal cavity, are widely available (FIG. 14). A quick search of online catalogs (e.g., Temu.com or Amazon.com) for “saline rinse bottle” will reveal multiple illustrations of such devices, generally in the range of $4 to $15 per unit. The unit may be sold together with packages of salts (sodium chloride (39 parts), sometimes with sodium bicarbonate (1 to 2 parts) to balance pH, for making the irrigating solution. The bottle's capacity is generally ˜240 to 300 mL, but 60 ml bottles are also available. Compression of the plastic bottle delivers a jet or plume of saline solution into one nostril, then onto the nasal cavity, then out the other nostril. The plume, like a garden hose on the nasal cavity surfaces, removes debris such as dirt, allergens, snot, encrustations, or dried mucus and phlegm. Some of the bottles, with a L-shaped neck, operate on gravity pressure have a button at the base for activating solution outflow. These plastic irrigation bottles may also be used as drug delivery units for a TRPM8 cooling agonist onto the surfaces of the nasal cavity and sinuses.

Measurement of Treatment Efficacy. Most studies on efficacy in the treatment of rhinitis have relied on patient-reported outcomes in the form of questionnaires. Objective measurements of nasal cavity volume by acoustic rhinometry or nasal airflow resistance by manometry have not shown a good correlation to the principal symptomatic complaint of stuffiness, congestion, or obstructed breathing. Here, we measure the patient's verbal response to treatment and the weight of nasal secretions as indices of treatment efficacy.

A top-loading balance, sensitive to 1 mg, was used to measure phlegm secretions from the nose. A pre-weighed tissue paper was tared to zero, and after blowing into the tissue, the amount of exudate was recorded from the balance display. This measurement is an objective sign of nasal inflammation. Microbalances, sensitive to 1 mg, are now available online for less than $20. Conventional laboratory balances with similar capabilities are listed for $2000+ each but are not suitable for patient monitoring of phlegm secretion. The use of an inexpensive method for quantifying phlegm output as an indicator of nasal pathology is part of the inventive steps.

Mechanisms of Action. In the past 20+ years, scientists have realized that cooling is a molecular event conveyed by dedicated nerve fibers containing TRPM8 (an integral membrane protein receptor). These nerve fibers fire when tissue temperatures drop below 25° C., stimulated by the negative heat flux or chemical agents. The cooling effect is modality-specific, and its anatomic circuitry has its own set of cables. TRPM8-mediated cooling is the physiological basis for “cool esthesia.” TRPM8 agonists replicate a negative heat flux sensation from the nasal membranes. Surprisingly, the studies here show that when these molecules are applied onto the nasal cavity surfaces, excess phlegm production dramatically declines. The experiments for finding an effective molecule and the appropriate delivery parameters are described here.

Inflammation is the reaction of vascularized living tissue to local injury (Cotran et al., Pathologic Basis of Disease, 1989, 4^{th} Edition). The cardinal signs expressed in inflamed tissues are redness (rubor), and heat (calor), due to increased blood flow, swelling (tumor) due to leakage of blood contents into tissues, and pain (dolor) due to activation of nerve endings in the injured tissue. The loss of function (functio laesa) resulted from tissue dysfunction. The injury of inflammation is mainly due to an increase in vascular permeability in the microcirculation. Substances released by injured tissues called inflammatory mediators, such as histamine enlarge gaps in postcapillary venules causing leakage of blood contents into tissues. The inflammatory cascade promoting excess fluids entering the interstitial spaces block gas exchange, distort tissue architecture, and impair tissue nutrition. In severe injuries, vascular leakage may persist for hours or days until thrombosis occurs, or the vessels rejuvenate. Inflammation is a dynamic process as the injured tissue seeks to defend, repair, and heal itself. In some instances, dysregulated inflammation itself becomes a source of further tissue injury, especially if the immune system is activated.

Anti-inflammatory drugs were in the clinic before mechanisms of drug action became understood. For example, non-steroidal anti-inflammatory drugs (NSAIDs), such as aspirin for pain relief, and corticosteroids, were used in rheumatoid arthritis before the cellular actions of prostaglandins and phosphorylases were known. Today we think of drugs as specific agonists, antagonists, enzyme inhibitors, or scavengers of inflammatory mediators. Usually, each drug is designed to work one-on-one against substances that promote inflammation. However, the efficacy of a single entity may be limited if more than one mediator acts during tissue injury. For example, an antihistamine works against the histamine released in seasonal allergic rhinitis but is less effective against non-allergenic rhinitis wherein histamine is one of many inflammatory mediators. An agonist, a term used to describe a chemical that activates biological events, is more effective than an antagonist if it can suppress convergent processes initiated by more than one inflammatory mediator. An example of a versatile agonist is hydrocortisone and related anti-inflammatory steroids, which are useful for treatment of rheumatoid arthritis, asthma, atopic dermatitis, and other inflammatory conditions.

It is proposed here that cooling agonists suppress a convergent site of inflammation. Physical lowering of temperature, for example, cooling with cold water or ice, is known to have potent acute anti-inflammatory properties. When applied to injured tissues, ice or an ice pack will stop the pain and swelling of traumatized or burned tissues. The mechanisms for how physical cooling stop acute inflammation are not known. If a chemical can mimic the effects of ice on acute inflammation, it will most likely act on a convergent point of the inflammatory cascade. Imagine the injured tissue as a parched grassland on fire (inflamed). Dousing the field with water is a general anti-inflammatory action. Making the field wet may also dampen the chances of added conflagration.

The versatile effects of a topical cooling gel (Intrinsic IB) on the skin of healthy volunteers were recently published by the applicant (Lee, S., Wei, E. T., et al. Histamine- and pruritogen-induced itch is inhibited by a TRPM8 agonist: a randomized vehicle-controlled human trial. Brit. J. Dermatol. 190 (6), 885-894, 2024. https://doi.org/10.1093/bjd/ljae054). A cooling agent inhibited itch induced by five pruritogens. This study show that cooling agents act on a convergent point of noxious stimuli.

Selection of Active Ingredient. The applicant has investigated cooling agents, that is, chemicals that produce cooling sensations without a change in tissue temperatures. Early in 2012, he noticed that the isopropyl esters of p-menthane glycine and p-menthane D-alanine had prolonged cooling activity when applied to the ocular margins (Wei, 2012). Subsequently, he discovered that diisopropyl analogs of phosphoryl alkanes produced refreshing coolness Yang et al. (2017). The isopropyl group appears to have specific anchoring sites on the TRPM8 receptor, which mediates sensations of coolness. Significant activity is lost if the isopropyl groups of phosphoryl alkanes change to sec-butyl. The studies of Xu et al. (2020) describe robust coupling energy between the isopropyl group on the cyclohexane ring of I-menthol and the L843 (leucine) and 1846 (isoleucine) residues of TRPM8. This “pocket” site for the “isopropyl legs” may be essential for prolonged TRPM8 receptor activation.

Molecules of Formula 1 are N-alkyl-diisopropyl-butanamides and first described in Roswell D G et al. Acyclic carboxamides having a physiological cooling effect. U.S. Pat. No. 4,296,255. Oct. 20, 1981. The molecules have a total mass of about 200 Daltons (FEMA 4557 has a mass of 199.12 Daltons). Synonyms for FEMA 4557 are WS-27 and N-ethyl-2,2-diisopropyl butanamide (CAS No. 51115-70-9). In studies described here, FEMA 4557 exerts a robust anti-inflammatory activity on skin at topical concentrations of ≥5% wt/vol and a total dose of several mg. FEMA 4557 stops pain and itch and exhibits unusual healing properties on injured tissues. FEMA 4557 applied to the inflamed nasal membranes stops the stuffiness of rhinitis and inhibits the secretion of phlegm. This occurs at ≤1.5% wt/vol. FEMA 4557 may be a prototype of a new class of anti-inflammatory agonists for nasal inflammation. Formula 1 shows the structure of the preferred embodiment, FEMA 4557.

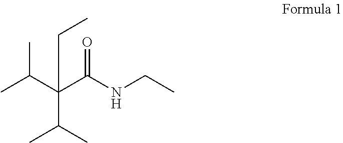

As of 2014, 30+ GRAS/FEMA cooling agents are listed (Leffingwell, 2014). The term GRAS means generally recognized safe (GRAS) and approved of use as a food additive or flavoring agent (Smith, 2009). On this GRAS list two compounds FEMA 3804 (WS-23) and FEMA 4557 (WS-27) have adjacent isopropyl groups. As shown below, FEMA 3804 (WS-23) is too short-acting to be useful for the nasal cavity but FEMA 4557 (WS-27) has a prolonged duration of cooling without irritancy on skin or nasal membranes, and, more surprisingly, FEMA 4557 is a potent inhibitor of acute inflammation. The relatively small molecular size of FEMA 4557 may facilitate dissolution and distribution to target surfaces, especially in inflamed tissues

The anti-inflammatory effects of FEMA 4557 were manifested in:
inhibition of the skin's response to insect bites (fleas and a bumble bee sting),
reducing the signs of skin irritation in a case of canine skin allergy,
reducing itch in severe urticaria, and
reducing pain and heat-induced swelling from a cigar lighter burn on the hand.
Inhibition of the symptoms and signs of rhinitis.

The preferred embodiment, FEMA 4557, exerted an immediate, profound, and unexpected anti-inflammatory drug action not seen with related substances. FEMA 4557 rendered the immediate injury reactions of pain, irritation, burning sensations, congestion, and itch less imperative. The subject could cope with the injury. Swelling and redness went down, and healing was rapid. This rapid tissue response to local injury was unusual and occurred after topical application.

Evolution of Cooling Agents from Menthol to FEMA 4557 Wilkinson Sword (WS) scientists tried to improve menthol 50+ years ago and made 1200+ analogs. Watson et al. (1978) summarized the results in a classical paper. The WS effort ended in 1980. Leffingwell and Rowsell re-visited and updated this subject in 2014; by then, thirty-two cooling agents were in the FEMA (Flavors, Extracts Manufacturing Association) database. FEMA-designated WS agents are GRAS (generally recognized as safe) and preferred for laboratory screening because FEMA has collected safety data. Some WS agents are assigned a FEMA number, periodically reviewed, and information posted online. Thus, the preference is for the FEMA label over the WS label.

The first intended use of WS analogs was for after-shave lotions (skin applications), but this shifted to cooling of the oral cavity. Today, metric tons per year of WS-3, WS-5, WS-12, and WS-23, together with menthol, are used as cooling ingredients in toothpaste (˜45%), chewing gum and confectionery (˜45%), and skin cosmetics and toiletries (˜10%). Recently, WS-5, WS-23, and FEMA 4557 (in China) entered the market as cooling substances in liquids for vaporization in e-cigarettes. However, no WS compounds ever became a therapeutic agent. FEMA 4549 and FEMA 3784 (menthoxypropanediol) were tested in clinical itch trials but did not enter development. The “WS” designations for WS-3, WS-5, WS-12, and WS-23 are familiar from past usage. But the preferred term is now the FEMA number, which has an extensive library online.

The first screening tests conducted in this discovery to assess candidates that produce refreshing cooling in the oral cavity. Test agents, prepared as 15 mg of white crystals, were placed on the dorsal surface of the tongue. The chemical structures and identifying characteristics are in Table 1. Surprisingly, FEMA 4557 (Formula 1, N-ethyl-2,2-diisopropylbutanamide, WS-27) stood out as a potent agent producing stinging, freezing, and painful cold on the throat. Related cooling agents were not freezing cold, except for WS-23, but WS-23 was too short-lived for practical use.

TABLE 1
Chemical structure of FEMA 4557

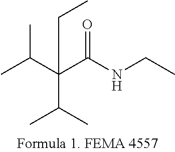

### FEMA 4557 Used for Topical Applications

After the oral cavity screen, FEMA 4557 was formulated into a topical gel and applied to the skin surface. This 4557-gel effectively treated skin dysesthesia in subjects with insect bites, skin allergies, and thermal injury. Related FEMA agents, such as FEMA 3804 (WS-23), WS-5, and WS-12, were ineffective under similar test conditions. The doses for FEMA 4557's topical activity on the skin were much higher than doses used commercially in the oral cavity. However, no tissue irritation or inflammation appeared on the skin, despite concentrations of >5% wt/vol. The average use levels (ppm)/average maximum use levels (ppm) for FEMA 4557, at which the FEMA Expert Panel based its judgments that the substances are generally recognized as safe (GRAS), is 3000/6000 ppm (or 0.3 to 0.6%) for FEMA 4557 in chewing gum, and 1000/1500 for confectionery frostings and hard candy. The skin tests confirmed and revealed the therapeutic value of FEMA 4557 as a topical medicament.

An example of a topical gel formulation of FEMA 4557 consisted of 8 to 16% wt/vol of FEMA 4557, 2% ethanol (or 1,2-propanediol) as a solvent, and 0.5% carbomer 980 as a gelling agent. L-Menthol may be included as a dispersing agent, penetration enhancer, and as an initiator. When applied topically, this gel will stop the irritation of an insect sting for 3+ hours with an onset of less than 3 minutes. In summary, a known flavoring agent, used primarily as an agent in the oral cavity, was found to be an effective pharmaceutical ingredient for topical application on skin.

Chemistry of Cooling Agents and Terminology. The prototype cooling agent is I-menthol, which occurs in plant species of the Mentha family. L-Menthol is one of eight enantiomers and has the greatest cooling activity. The WS compounds most widely used in commerce (WS-3, WS-5, and WS-12) are p-menthane carboxamides. p-Menthane has a compact cyclohexane ring with the steric configuration of I-menthol. WS scientists also discovered “acyclic amides” that exerted cooling actions (Rowsell et al., 1981). The acyclic amide, WS-23 and WS-24 were commercialized in China for liquids used in electronic cigarettes (Wu et al. J Appl Toxicol. 41 (11): 1826-38, 2021). These compounds are “acyclic” because they do not contain the cyclohexane ring. The applicant described the properties of the cooling agents icilin and 1-dialkylphosphorylalkanes, represented by 1-diisopropyl-phosphorylheptane (synonyms: Cryosim-1, DIPA-1-7: CAS No.) which do not contain cyclohexane.

The monoterpenoid sensates (e.g., menthol, eucalyptol, camphor), p-menthane carboxamides, dialkylphosphorylalkanes, icilin analogs, and acyclic amides can generate an infinite variety of analogs. However, which will work in the clinic? In practice, one cannot test thousands of compounds for clinical utility. The number of lead candidates must be sifted and manageable.

Structure-Activity and Isopropyl Legs In previous studies, Wei (2012) found that the isopropyl esters of p-menthane carboxamides had prolonged cooling activity on ocular margins, by contrast to methyl, ethyl, and butyl esters. Subsequently, he described 1-diisopropylalkylphosphorylalkanes as potent stimulators of the TRPM8 receptor. These molecules, called “Cryosims,” are effective for urticarial rashes and scalp dermatitis in human subjects (Jung et al., 2021; Kang et al., 2022). FEMA 4557 and FEMA 3804 (WS-23) are similar structures (Table 2). Both have prominent isopropyl legs in close juxtaposition to the hydrogen-bonding oxygen atom. FEMA 4557 produced a potent stinging cold on the tongue, but not FEMA 3804. The extra methyl group (marked as *** in Table 2) of FEMA 4557 may fit better into the receptor pocket. Changing the N-alkyl substituent to n-propyl, isopropyl or cyclopropyl may further alter the duration of action.

Xu et al. (2020) conducted a thorough analysis of the binding of I-menthol to the mouse TRPM8 receptor. They noted the robust coupling energy between the isopropyl group on the cyclohexane ring and the channel based on the Kd values. For the critical binding amino acid residues, they stated that the highest coupling energy values for the “isopropyl leg” were at L843 (leucine) and 1846 (isoleucine). In contrast, the V775 (valine) residue outside the binding pocket had smaller coupling energy with the isopropyl legs. Y745 (tyrosine) and F839 (phenylalanine) were within the binding pocket, but they exhibited smaller coupling energy than 1846 and L843. The results of Xu et al. suggest that the isopropyl legs point downward and stand on residues L843 and 1846 to facilitate channel activation.

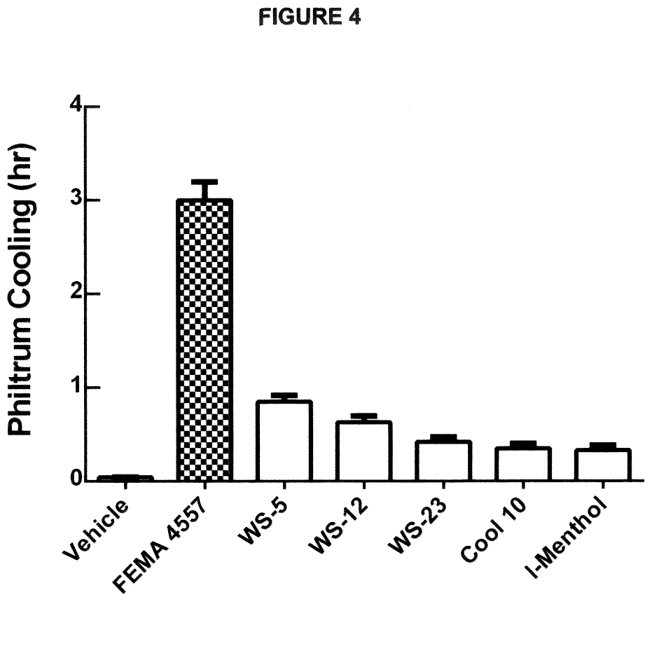

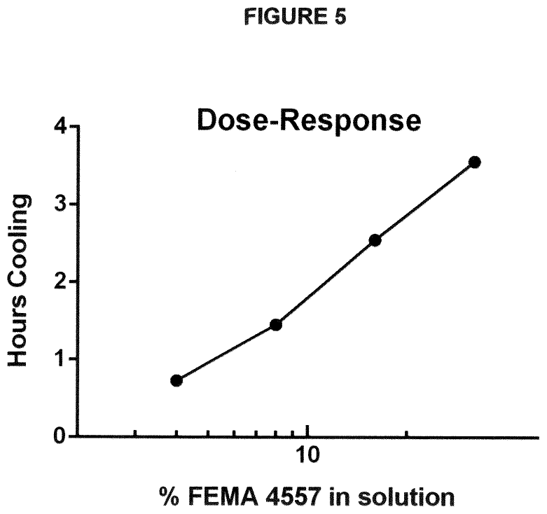

The diisopropyl “legs” of FEMA 4557 made it attractive for further screening. Most data on structure-activity relationships of p-menthane carboxamides are for cooling activity in the oral cavity and for in vitro TRPM8 receptor activation (EC₅₀ values). However, we find that FEMA 4557 had low potency on TRPM8 but an unexpected and surprisingly long duration of cooling action when applied to the philtrum skin. The extra methyl group on FEMA 4557, marked as (***), and which is not on WS-23, may act as a “hook” or “coat-hanger” to trap the molecule at the receptor pocket and prolong occupancy and duration of action. FIG. 4 show the relative activity of FEMA 4557 to other compounds. FEMA 4557's potency on the philtrum test is exceptional. The dose-response relationship of FEMA 4557 on the philtrum skin is shown in FIG. 5.

TABLE 2
Chemical structures of FEMA 3804 and FEMA 4557

| FEMA 3804 (WS-23) | FEMA 4557 (WS-27) | *** (hook) on FEMA 4557 |
|---|---|---|
| 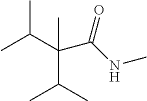 | 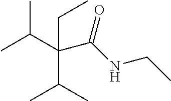 | 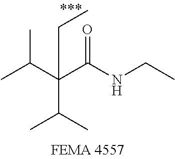 |

TABLE 3
Isopropyl legs in the structure activity analysis of cooling agents with
the properties of freezing, painful cold.

| FEM | | | | Effects of 15 mg |
|---|---|---|---|---|
| A No. | CAS No. | Synonyms | Structure | on tongue |
| 2665 | 2216-51-5 | I-Menthol | 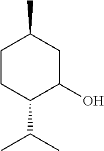 | |
| — | 1487170- 15-9 | Cryosim-1 | 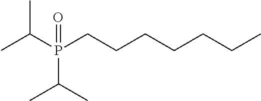 | stinging, painful cold |
| | 1401555- 39-2 | Ax-8 p-Menthane-Gly- OiPr Wei US 8,426,463. | 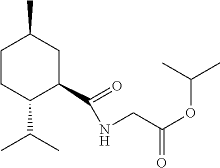 [SMILES: CC(C)OC(=O)CNC(=O)[C@@H]1C[C@H](C)CC[C@H]1C(C)C] | pleasant cooling in oral cavity, ~27 min, no freezing cold or pain, or sting |
| 4557 | 51115-70-9 | WS-27, N-ethyl-2,2- disiopropyl butanamide, N,2-diethyl-3-methyl- 2-(1-methylethyl)- butanamide | 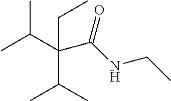 | stinging, painful cold |
| 3804 | 51115-67-4 | WS-23 | 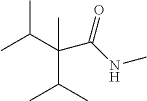 | |

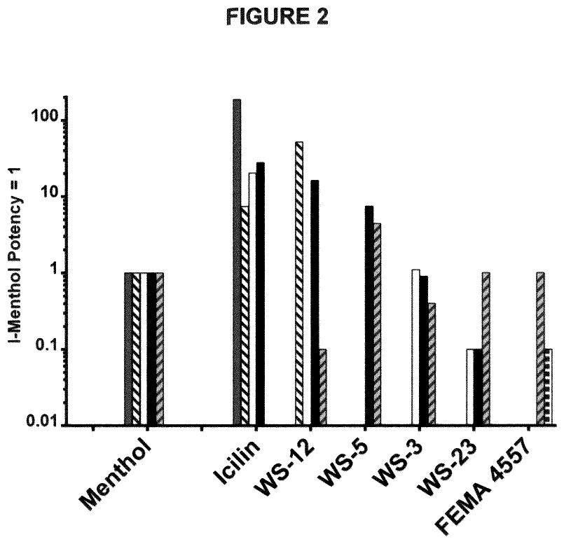

TRPM8 Receptor Potency, the EC₅₀ In 2002, groups led by David Julius and Ardem Patapoutian (Nobel Laureates of 2021) showed that menthol and icilin, both cooling agents, acted on a neuronal membrane protein later called the TRPM8 receptor. A receptor assay is a standard rapid screening tool. For example, in a 384-plate assay, ten compounds can be tested to yield dose-response data. The in vitro TRPM8 assay generates an EC₅₀ (median effective concentration) for an agonist to indicate potency. However, the quantitative correlation between the EC₅₀ on TRPM8 and cooling sensation is not exact. The science is “roughly” predictive, but each agonist has considerable uncertainty and variability on the chosen target surface. The EC₅₀ values have scientific panache but do not give information on the duration of action or the quality of the sensation. Table 4 summarizes the results of TRPM8 EC₅₀ (see FIG. 2).

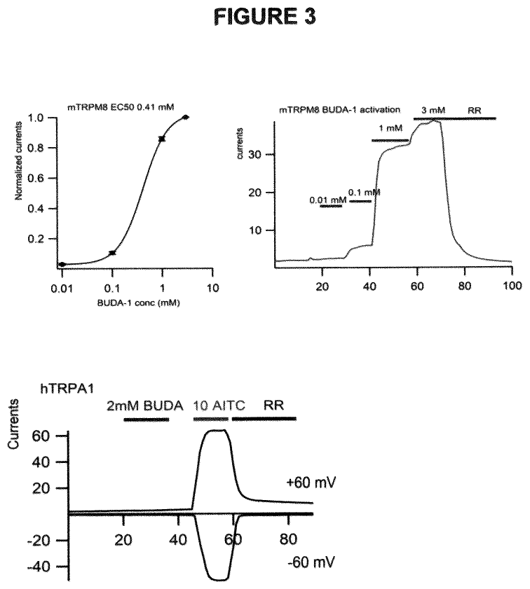

The TRPM8 median effective dose (EC₅₀) of various agents are compiled and shown in Table 4 and FIG. 2. The consensus is that icilin and WS-12 are the more potent agonists, followed by WS-5>WS-3˜I-menthol. The acyclic amides WS-23 and FEMA 4557 are significantly less active than I-menthol. The applicant provided the test substances used in the publications by Mckemy et al., Bodding et al., and Sherkheli et al., as used in the Table and Fig. Further experiments by the applicant confirmed the weak activity of FEMA 4557 on TRPM8 (10% the activity of I-menthol) and the absence of activity on TRPA1. The bioassay data of FEMA 4557 (labeled as BUDA) in TRPM8 and TRPA1, were generated in the laboratory of Prof. Seok-Yong Lee at Duke University (FIG. 3). The EC₅₀ of FEMA 4557 is at 0.41 mM or about 1/30× that of I-menthol. FEMA 4557 was not active in cells transfected with TRPA1. A positive TRPA1 control AITC (allyl isothiocyanate) was active on TRPA1.

The results of Johnson et al. (Ref #3) differ most from the data of others. Inspection of Johnson's data shows the numbers do not fit the Hill Equation criteria for an EC₅₀ measurement. In a Sigmoid Curve, it is essential to have data points above and below the median value, but Johnson's data does not have it. Overall, one can conclude that the acyclic amide coolants, WS-23 and FEMA 4557, are less potent (e.g., ⅕ to 1/10) than menthol. The result on FEMA 4557 was from Prof Lee's laboratory at Duke University (Prof Lee is the world's foremost expert on TRPM8, having published three papers in “Science” on this subject in the past four years.) Thus, the surprising high potency of FEMA 4557 on the philtrum test is unexpected (FIG. 4).

TABLE 4
EC50 of Cooling Agents Tested on TRPM8, See FIG. 3 for graph

| | EC50 | | EC50 | | EC50 | | EC50 | | EC50 |
|---|---|---|---|---|---|---|---|---|---|
| Ref#5 | μM | Ref#1 | μM | Ref#2 | μM | Ref#6 | μM | Ref#3 | μM |
| icilin | 0.36 | Icilin | 0.2 | WS-12 | 0.2 | Icilin | 7 | WS-5 | 0.5 |
| menthol | 67 | WS-3 | 3.7 | CPS-113 | 1.2 | WS-12 | 12 | FEMA- | 1.6 |
| | | | | | | | | 4557 | |
| | | menthol | 4.1 | Icilin | 1.4 | WS-5 | 26 | menthol | 2.2 |
| | | Cool 10 | 6.0 | CPS-369 | 3.6 | CPS- | 65 | WS-23 | 2.2 |
| | | | | | | 369 | | | |
| | | WS-23 | 44 | WS-148 | 4.1 | menthol | 196 | WS-3 | 5.9 |
| | | | | WS-30 | 5.6 | WS-3 | 210 | WS-12 | 22.1 |
| | | | | menthol | 10.4 | WS-23 | 1500 | WS-24 | 34.5 |

Reference # in Table 4 and plotted in FIG. 3
1. Behrendt, H-J. et al., Characterization of the mouse cold-menthol receptor TRPM8 and vanilloid receptor type-1 VR1 using a fluorometric imaging plate reader (FLIPR) assay. Brit. J. Pharmacol. (2004) 1-9.
2. Bödding M, et al.. Characterisation of TRPM8 as a pharmacophore receptor. Cell Calcium. 2007; 42(6): 618-28.
3. Johnson S, et al., Trigeminal Receptor Study of High-Intensity Cooling Agents. J Agric Food Chem. 2018; 66(10): 2319-23.
4. Leffingwell J, Rowsell D. Wilkinson Sword cooling compounds: from the beginning to now. Perfume & Flavorist 39: 34-44, 2014. Provides a similar analysis of published data.
5. McKemy D D, Neuhausser W M, Julius D. Identification of a cold receptor reveals a general role for TRP channels in thermosensation. Nature. 2002; 416(6876): 52-8.
6. Sherkheli M A, et al., Characterization of Selective TRPM8 Ligands and their Structure. Re 2010; 13(2): 242-53.

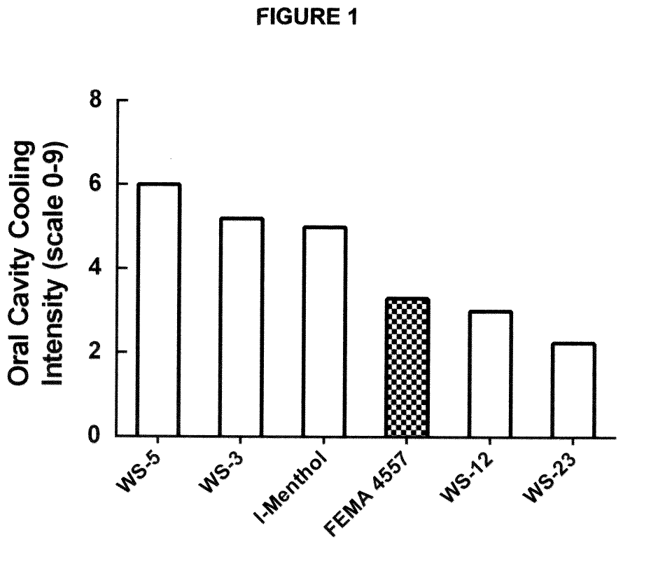

See FIG. 1. Cooling intensity of agents tested in the oral cavity at 100 ppm in 5% sucrose. Cooling intensity was self-rated on a scale of 0 to 9 units. Data are from of Sonya Johnson et al., 2017. The rank order of potency of WS-5>WS-3˜I-menthol>WS-12>WS23 agrees with compilations published by Leffingwell (2014) and by Erman (2007).

The sensory neurons of the skin, studied in isolated dorsal root ganglia neurons, have TRPM8 as the primary protein for detecting cold. However, in about 20% of such labeled neurons, the TRPM8 co-exists with TRPV1 in the same neuron. Co-activation of such TRPM8/TRPV1 neurons may give the sensation of icy, stinging, painful cold. TRPA1 is another TRP channel that co-exists with TRPM8 and TRPV1. Johnson et al., 2017 reported that FEMA 4557 activates TRPA1, but we cannot confirm these results. The precise target receptor of FEMA 4557 for anti-inflammatory activity requires further study.

Philtrum Skin Test for Quantifying Topical Bioactivity John Gaddum, one of the pioneers of modern pharmacology, established the concept of Bioassay and the EC₅₀. He was fond of pithy sayings. Some memorable quotes are:
“A branch of science comes of age when it becomes quantitative.”
On research. “Enthusiasm is not enough.”
“The pharmacologist has been a ‘jack of all trades’, borrowing from physiology, biochemistry, pathology, microbiology, and statistics—but he has developed one technique of his own and that is the technique of bioassay.”

In the search for a molecule that will suppress inflammation after topical application, it is essential to have the right tests, even if the circumstances of injury are by accident. Wilkinson Sword in their program for finding better menthol analogs, tested substances by impregnating them on filter paper and applying the paper to the tongue of volunteers. The threshold for cooling on the tongue surface was rated with “+” signs to signify potency and later quantified as “μg” on the tongue. The data were “semi-quantitative” and there is overlap in the potency of many analogs. As WS compounds became widely used in the 1990s in toothpaste, chewing gum, mouthwash, and confectionery, there was a need to re-design the bioassay for the oral cavity. The WS method was replaced by the “hold and spit” method. In this assay, a test substance is suspended or dissolved in a liquid, and the liquid held for 30 sec in the mouth and then expectorated. Sometimes, sucrose is added to the test liquid. The sensory effects are recorded by the subject and “cooling intensity in the oral cavity” is rated on a scale of 0 to 9. No attempt was made to relate oral cavity cooling to effects on skin. The general consensus was that WS-5 gave the most intense cool, followed by WS-3 and I-menthol, and then WS-12. WS-23 and WS-24 were considered less potent.

Wei (2012) proposed the use of the skin on the philtrum as a test site for cooling activity of chemicals. The philtrum is the midline groove above the upper lip border and vermilion and is the drug application site. The gel is placed on the philtrum with a cotton-tipped applicator up to the nasolabial folds and sometimes, but not deliberately, on the lower nostrils (subnasale). Cold receptors densely innervate the philtrum skin, second only to the surfaces of the eyeball, nasal surfaces, and anogenitalia. The lips also have a high density of “cold spots.” This is called the philtrum assay. The test substance is dissolved in a carrier, such as solvent, gel, or ointment and applied to the philtrum, a surface area of ˜10 to 12 cm2. The subject records sensations on the philtrum and environs on a scale of 0 to 3, as: 0=no changes in sensation, 1=a slight feeling of coolness, cold or tingling, 2=a clearcut feeling of coolness or cold and 3=a robust feeling of coolness and cold. The intervals for measurements are usually 0, 1, 3, 5 min after application, and at then 5 min or longer intervals thereafter until two successive zeros are obtained.

Test substances are applied to the philtrum using a cotton-tipped applicator (Puritan Cotton-tipped applicators, or Q-Tips). The applicator tip is dipped into a reservoir bottle containing the liquid or semi-liquid (gel) test substance. The composition adhering to the cotton (about 0.2 mL) is put on the skin. A subject can practice the method in front of a mirror with a high-intensity light source.

TABLE 5
Cooling agents screened for cold activity in the oral cavity. The FEMA
compounds have been evaluated by an Expert Panel and is generally recognized as safe
(GRAS) for its intended use.

| FEMA | CAS | | | Effects of 15 mg |
|---|---|---|---|---|
| No. | No | Synonyms | Structure | on tongue |
| 4557 | 51115- 70-9 | WS-27, N- ethyl-2,2- disiopropyl butanamide FEMA 4557 | 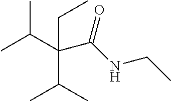 | stinging, painful freezing cold in throat, >35 min |
| 3804 | 51115- 67-4 | WS-23 FEMA 3804 | 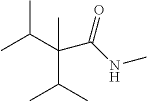 | painful cold in throat, ~18 min |
| 3455 | 39711- 79-0 | WS-3 FEMA 3455 | 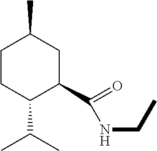 | pleasant cooling in oral cavity, ~8 min, no freezing cold or pain, or sting |
| 4681 | 68489- 09-8 | WS-12 FEMA 4681 | 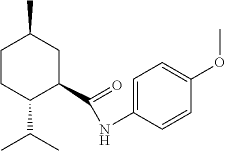 [SMILES: COc1ccc(NC(=O)[C@@H]2C[C@H](C)CC[C@H]2C(C)C)cc1] | pleasant cooling in oral cavity, ~20 . min, no freezing cold or pain, or sting |
| 4309 | 68489- 14-5 | WS-5 FEMA 4309 | 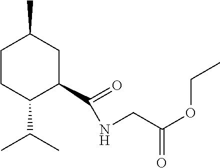 [SMILES: CCOC(=O)CNC(=O)[C@@H]1C[C@H](C)CC[C@H]1C(C)C] | pleasant cooling in oral cavity, ~12 min, no freezing cold or pain, or sting |

TABLE 6
Results with Test Substances, see FIG. 4 graph.

| | % Conc. | Duration | |
|---|---|---|---|
| Chemical | wt/vol | (min) | Comments |
| FEMA | 8 | 180+ | icy cold sensations, prolonged cooling, anti-itch |
| 4557 | | | effects on flea bites and other skin irritations such |
| | | | as pollen allgergies, qualitatively and |
| | | | quantitatively different from the other test analogs, |
| | | | no irritation of the skin was observed with FEMA |
| | | | 4557 and concentrations of up to 12% |
| | | | weight/volume applied to the philtrum skin. The |
| | | | average time of cooling for 2, 4 and 8% |
| | | | concentrations were 50, 95 and 175+ min, |
| | | | respectively. |

Other compounds tested on the philtrum assay at 8%.

| | | | |
|---|---|---|---|
| WS-23 | 8 | 25 ± 3 | short duration of action, no overt cold, but more |
| | | | coolness |
| WS-12 | 8 | 38 ± 4 | pleasant cool sensations on skin |
| WS-5 | 8 | 51 ± 4 | pleasant cool sensations of skin |

FIG. 4. Cooling agents, prepared as an 8% wt./vol. gel, was tested topically on the philtrum skin assay. The duration of cooling was recorded (min on y-axis). The startling activity of FEMA 4557, cooling for ˜3 hr, was unexpected and surprising. FEMA 3784 is also known as Cool 10 or menthoxypropanediol.

Note that where R=ethyl, the compound Is FEMA 4557 and has been described in Roswell, Hems and Spring, U.S. Pat. No. 4,296,255 Oct. 20, 2021. The isopropyl, n-propyl cyclopropyl, or acetyl analogs have not been synthesized and tested. There is reason to believe that these analogs may be more potent than the ethyl analog. The extra methyl group forming the “hook” or “coat hanger” on FEMA 4557 is critical for the practice of this invention. The hook is ethyl in FEMA 4557, but can be modified to n-propyl, or i-propyl to increase activity, as shown in Formula 2. Permeation through the stratum corneum is not an essential factor for success because FEMA 4557 is much more active than WS-23, in spite of having an extra methyl group.

TABLE 7
Structure of FEMA 4557 Analogs Contemplated in this Discovery

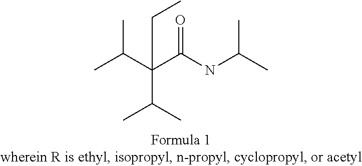

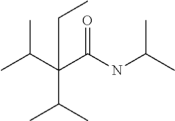

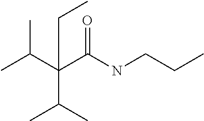

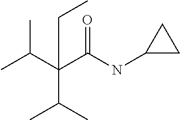

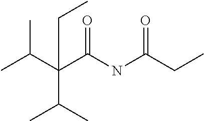 [SMILES: CCC(=O)NC(=O)C(CC)(C(C)C)C(C)C]

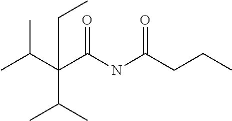 [SMILES: CCCC(=O)NC(=O)C(CC)(C(C)C)C(C)C]

Formulation, Excipients, and Initiators The formulation of skin care products is a secretive process. Success may depend on slight changes in the selection or levels of ingredients without an obvious explanation for differences. Here, the choice of the carbomer polymer for gelling is straightforward, and varying the concentration will determine the appropriate gel viscosity. In formulations with FEMA 4557, anti-inflammatory steroids, hormones, or emollients can be mixed in adjuncts. Other consideration in formulation are:
The drug should be potent, cost-effective, and receptor-selective to avoid side effects.
The formulation for delivery should be easy to prepare.
The technology should be original and allow patentability for development.
The quality of sensation should be refreshingly cool or cold, without irritation or pain.
Identifying the ideal chemical class and molecule is a matter of continued and persistent experiment. When the criteria defined above, 95+% of known cooling molecules will fail! However, one N-alkyl, 2,2 diisopropyl butanamide structure, e.g., FEMA 4557, meets these criteria of successful implementation.

### Therapeutic Applications of FEMA 4557 in Inflammations of the Nasal Mucosa

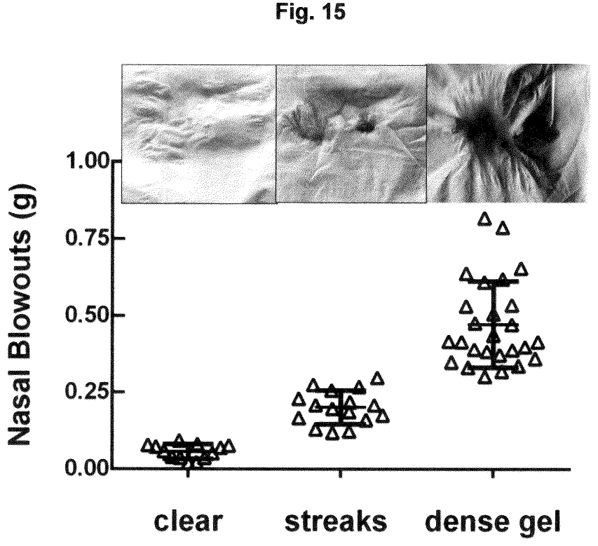

The nasal cavity mucosa is a complex system. When there is inflammation (rhinitis), several components are activated and sentient.
The perception of impaired breathing can be evoked by pinching the nasal ala, the outer, fleshy, and cartilaginous structure that forms the wing-like sides of the nostrils. Pinching is a physical method to produce the sensation of stuffiness. A heat load in the nasal cavity will also cause stuffiness. For example, a facemask in hot weather will cause a heat load and stuffiness. Stuffiness is relieved by a negative heat flux. Movement of cold air across the nasal cavity surface, e.g., with an air conditioner, will relieve stuffiness. Stimulation of the TRPM8 receptor on nerve fibers with an agonist mediates coolness via a similar mechanism. Stuffiness is the main complaint of patients with rhinitis because this sensation at night will interfere with sleep. The cooling effects of FEMA 4557 were clear, and unmistakable, akin to stepping out into 40 to 55° F. air and taking a deep breath. The stuffiness was relieved.
Mucus is secreted from glands of the nasal mucosa. Mucus is a transparent white protein when secreted but when mixed with exudates and cell debris it adopts a slimey, viscous yellow gel called phlegm or gunk. When copious, It is dense and unpleasant to look at when blown out onto a wipe (FIG. 15). When phlegm dries, it becomes crusted and hardened, and called “snot.” Phlegm can physically reduce nasal cavity volume, but the patient with rhinitis typically clears the mucus by blowing the nose or using irrigation with water or saline. The phlegm in the nasal sinus cavity when extensive, give the opacity that appears in a CT scan. The weight of phlegm blown out onto a tissue paper provides a quantitative and dynamic index of nasal inflammation.

The nasal cavity must be kept clean, especially from deleterious phlegm or gunk, which is a mixture of excess mucus, secretions, and debris of inflammation. Rhinologists know that phlegm harms the nasal passages, and irrigation of the nose to remove phlegm is now familiar (Rogers et al. Resp. Care 52:1134-1146, 2007: Hill et al. Physiol. Rev. 102:1757-1836, 2022). This type of disorder affects up to 15% of the general population. Stopping stuffiness in rhinitis alone has value because it is comfortable. The gunk is the principal pathological feature. For inflamed nasal passages, a rapid and efficient medication to remove gunk can revolutionize rhinitis treatment. Approximately 250,000 surgeries are performed each year by rhinologists (in the USA). With new cryosurgical and electrolytic ablation of nasal nerves, surgery is likely to increase, but at about $25,000 per procedure. A simple 3- to 5-day course of nebulized FEMA 4557 to treat phlegm secretion, the worst consequence of rhinitis, may help save resources for managing rhinitis. One gram of FEMA 4557 at 2024 prices costs ˜$0.15 ($150 per kg). One gram is sufficient to create four bottles of NeilMed Sinus Rinsing solution. Each bottle may be sufficient to treat one patient with rhinitis.

Allergic rhinitis, vasomotor rhinitis, and chronic rhinosinusitis are generally viewed as long-term disorders (>12 weeks), with changes in the immune response to nasal injury. The results herein suggest that rhinitis may be treatable as an acute inflammatory disease of the nasal mucosa, much like a mosquito bite or a pustule. The treatment goal is to reduce phlegm production below 0.25 g per blowout event. The subject's nasal mucosa then begins to recover and resurrects to clean and refreshed breathing. This resurrection can become permanent and maintained. This change may occur within three to five days after treatment. Phlegm is the culprit for chronic pathology. Reducing phlegm production may halt and reverse the course of the disease.

FEMA 4557: Treating Nasal Cavity Inflammation with a Spray

Inflammation of the mucosa of the nasal cavity and sinuses is called rhinitis and sinusitis. About 15% of the general population suffers from rhinitis and ˜5% from rhinosinusitis. A prominent complaint of patients with rhinitis and sinusitis is “stuffiness,” or the subjective feeling of impaired airflow, loss of patency, and uncomfortable breathing. When the inflammation is severe, thick, purulent, and discolored nasal discharge may be present. Sinusitis is accompanied by facial pain, pressure or fullness, and changes in the sense of smell (hyposmia or anosmia). Radiological imaging, such as computer tomography (CT) scans, helps diagnose impaired sinus drainage. The sense of impaired breathing or stuffiness affects sleep and patients' quality of life.

The underlying triggers and mechanisms of rhinitis vary. The mucosae react to allergens, viral or bacterial infections, inhaled irritants such as odors, perfumes, and smoke, changes in temperature or humidity, hormonal changes, or occupational exposure to chemicals, dust, or fumes. Anti-inflammatory steroids and antihistamines treat allergic rhinitis but are less effective against non-allergic rhinitis. When drug treatments fail to control symptoms, surgery is an alternative. Endoscopic sinus surgery is a procedure to remove obstructions, inflamed tissues, and nasal polyps to facilitate drainage of the nasal passages. More recently, rhinologists have been using cryosurgical and electrolytic procedures to remove the nerves of the nasal cavity to treat recalcitrant rhinitis. As the patient base for rhinitis and sinusitis is large, there is a need for further innovation.

Study 1. In this first study, a specialized nebulizer with an electronically controlled compressor was used to deliver FEMA 4557 to the nasal cavity of subjects with rhinitis. A typical dosage regimen was a 1.0 to 1.5% wt/vol solution (10 to 15 mg/mL) of FEMA 4557 over a 3 to 5 sec delivery period. A dosage schedule of two to three daily activations for three to five days relieved symptoms and allowed the subjects to manage and control the rhinitis. No attempt was made to differentiate between delivered versus retained dose.

The severity of rhinitis was quantified by gravimetric measurement of secretions and exudates from the nasal cavity (FIG. 15). Subjects were instructed to blow into a pre-weighed paper tissue and record the weight of the tissue after blowing. The tissue was inspected and marked as clear, with streaks of exudates, or dense gel. The nasal blowouts fell into three distinct categories of weights that were easy to recognize. The relative unit weights (mean±s.e.m.) are clear, 0.06±0.01 g, streaks 0.20±0.01 g, and dense gel, 0.47±0.03 g. The weight of the blowouts provides an objective index of nasal cavity inflammation.

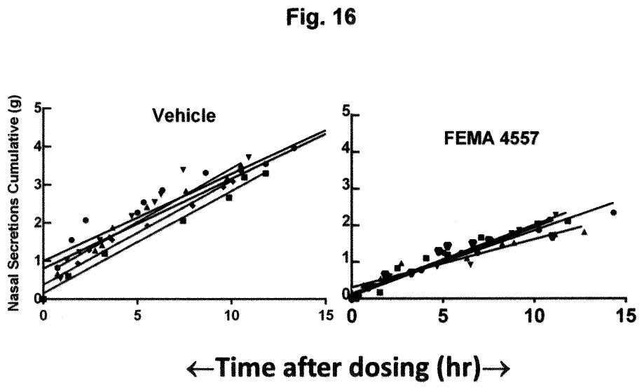

Nasal secretions were inhibited by topical administration of FEMA 4557, dissolved at 15 mg/ml in an ethanol, polysorbate 80, and saline mixture (FIG. 16). A Nasoneb® apparatus delivered FEMA 4557 into the nasal cavity (FIG. 13). The test substance was sprayed into the nasal cavity for 5 sec at t=0, 2, and 5 hr. The data points were fitted by linear regression (GraphPad Prism), and the 10-hr secretion was measured as (avg.±s.e.m). The results were vehicle 3.16±0.10 g and FEMA 4557 1.86±0.07 g, with n=6 and 5 observations per group, respectively. The nasal secretion-10 hr unit for FEMA 4557 was significantly lower (P≤0.01) than the vehicle when analyzed by the Kruskal-Wallis test for non-parametric data. The anti-inflammatory effect of FEMA 4557 on rhinitis is thereby demonstrated.

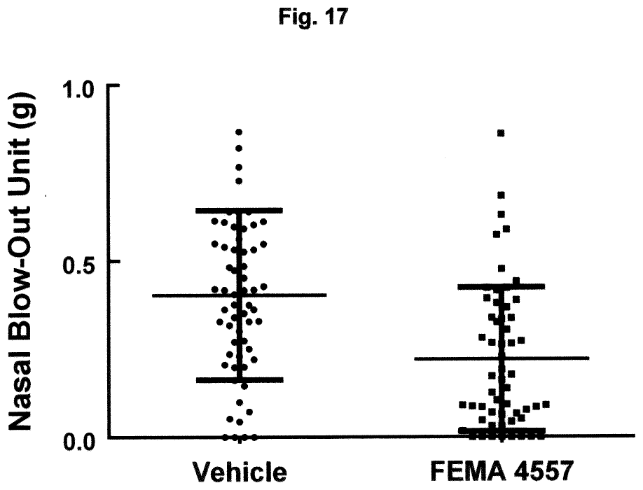

Inhibition of nasal secretions by topical administration of FEMA 4557, 15 mg/ml, delivered into the nasal cavity, is further illustrated by plotting the individual blowout units, in g per blowout FIG. 17. The test substances were sprayed into the nasal cavity for 5 sec at t=0, 2, and 5 hr. The “blowouts” (average±s.e.m.) for blowouts were vehicle 0.38±0.03 g and FEMA 4557 0.21±0.03 g, with n=61, and n=56 observations per group, respectively. When analyzed by the Kruskal-Wallis test for non-parametric data, the blowout units were significantly different (P<0.0001). Thus, the anti-inflammatory effect of FEMA 4557 is further confirmed.

Four individuals with rhinitis treated with the NasoNeb® spray two to three times per day for three days felt their nasal discharge and stuffiness were reduced to an extent such that they no longer wanted to participate in the experiment. These subjects pronounced that their rhinitis was “controlled,” and the condition did not bother them anymore. These subjects declared that any recurrence of symptoms could be managed by spraying on an “as-needed basis.”

FEMA 4557: Treating Nasal Cavity Inflammation with Nose Drops

Nose drops are used to treat conditions of the nasal cavity, such as nasal congestion, allergic rhinitis, and sinusitis. Topical medications are considered adequate, but nose drops use varies in clinical practice. Sprays are generally preferred to nose drops because of ease of administration and expectations of better distribution of the medication. The use of drops may be inconvenient for the elderly and young and requires extra instruction on the correct technique. Here, FEMA 4557 nose drops were tested on a subject with a severe cough due to allergic rhinitis and post-nasal drip. This is a condition caused by the trickling of excess phlegm into the pharynx.

Study 2. The subject, Robert, an 80-year-old male, had allergic rhinitis with the typical signs of vigorous sneezing, itchy eyes, and runny nose. During the spring, he experienced severe bouts of coughing, especially during the night when he was trying to sleep. He felt excessive phlegm forming and clogging his throat. The applicant was given several 10 mL plastic bottles containing FEMA 4557, a 10 mg/mL suspension in 1% ethanol/2% polysorbate 80, and saline vigorously mixed in a Flack Speedmixer. The subject was instructed to put one or two drops in each nostril, then pinch and release the nose with thumb and forefinger. This pinching maneuver helps disperse the drops into the medial turbinates.

Promptly after receipt, Robert began using it in the evenings. He had purchased Addipak® 5 mL sterile 0.9% NaCl solution vials, which he first used to clean out his nasal passages (and sometimes also took a steamy, hot shower before or after using the nasal rinses). Robert then shook the FEMA 4557 bottle provided and administered a drop into each nostril while sniffing in, holding/pinching his nose for a moment and sniffing more after nose release. He waited a moment to let the drops sink in. If the phlegm started to drop down in his throat, he would spit it out.

Soon after this treatment, Robert could sleep without coughing for 7 to 8 hours. He recommended these procedures to follow:
Take a shower, and let the steam help clear the nasal passages. Clear out as much phlegm as possible during the shower.
Clean out nasal passages with the Addipak™ saline solution. Clear out as much phlegm as possible.
Shake the nose drops bottle before using. Put a drop of the FEMA 4557 suspension in each nasal passage. Sniff the drop in, pinch the nose, and let go.

In Robert's experience, coughing stopped, and the airways cleared for a good night's sleep for 7 to 8 hours. This experiment is the first time in which nose drops with a cooling agent inhibit the mucus hypersecretion in the nasal cavity caused by allergic rhinitis. It successfully treats the “post-nasal drip” cough, also known as upper airway cough syndrome. Surprisingly, the drops work on cough when put directly on the oropharynx.

FEMA 4557: Treating Nasal Cavity Inflammation with a Rinse

Study 3. Saline irrigation methods are now widely available and can be found in online catalogs for treating rhinitis. Flexible plastic bottles usually hold about 240 to 300 ml of isotonic and PH-balanced saline or saline/bicarbonate solutions to generate a liquid plume into the nostrils by manual bottle compression. The volume per activation may be in the range of 2 to 12 mL and the pressure applied can be optimized to moisturize, rinse, or hose the nasal surfaces. A vigorous hosing can use up to ≥60 mL in one episode. Mehta (U.S. Pat. No. 6,520,384) described the NeilMed Sinus Rinse device in detail. The saline plume from the rinse bottle acts like a garden hose for loosening and removing debris on the nasal cavity and sinus surfaces. Lavaging allows the gunk on these surfaces to drain out via the nares and reduce inflammation of the mucous membranes. The methods using a NasoNeb nebulizer, nose drops, or rinse apparatus have better safety features than hand-held sprayers because droplet sizes are larger and do not become aerosolized to enter the lower airways.

FEMA 4557 rinse solution was formulated as 1 mg/ml in 1% ethanol/0.65% wt./volume saline. The FEMA 4557 solution delivered with a rinse was studied using a standard rinsing apparatus (Neilmed Sinus Rinse). Solutions were kept at ≥25° C. to avoid precipitation of FEMA 4557. A 30 sec to 1 min heating of a solution in a microwave is one method of keeping the solution warm. The recommended fill volume of this bottle is 240 mL of isotonic saline. By weight measurements, it was discovered that the average volume dispensed per activation was 5.6 mL±0.47 (s.e.m.) (4.6 to 6.5 mL, 95% CL). The average volume retained in the nasal cavity was 1.8±0.15 mL (34%). Thus, the average net dose was 1.8 mg of FEMA 4557 and 18 mg of ethanol. Surprisingly, this dosage regimen effectively reduced the rhinitis symptoms of stuffiness and excess phlegm. The four test subjects with rhinitis symptoms declared they had cool breathing and slept better at night. The rinse method of drug delivery was about ten times less expensive than the method using the NasoNeb device. The test subjects also felt it was easier to use than nose drops. The ˜2 mL delivered into the nasal cavity will likely distribute more to the OMC than a hand-held manual sprayer.

The case history of one participant may illustrate these experiments. A 48-year-old male entrepreneur with some background in biology volunteered for a self-experiment with FEMA 4557. His commercial interest in this technology led him to closely monitor this subject for at least four weeks. Here are some of his insights and suggestions.

“I developed allergic rhinitis about 25 years ago. On a golf course, my eyes suddenly became intensely itchy. I sneezed, had a runny nose, and coughed. This persisted, and my doctor gave me topical and oral antihistamines, then later inhaled steroids. These drugs helped a bit, but over the past ten years, my condition has worsened to both allergic and non-allergic rhinitis. I quickly react to odors, cleansers, and dust. Sometimes, first thing in the morning, when I get up, I put on a face mask to avoid sneezing. In business meetings, if I sneeze, blow my nose, or sound hoarse, people ask if I have a cold, which is embarrassing. My wife complained all the time about my snoring. The drugs no longer work. I saw a rhinologist about possible endoscopic surgery, but after a CT scan and MRI he said that there were no anatomic abnormalities to justify surgery. After trying the FEMA 4557 spray and rinsing solution and having detailed discussions with the inventors, I now have a good idea of how to control the nasal problems.

The fundamental problem of rhinitis is the excess secretion of mucus (or phlegm) that dries in or on the nasal cavity surface. The “streaks” of light-yellow mucus stick to the nasal hairs, form “crusts” in the nose, and coat the surface of the inner nose. Any dried crust, large or small, is felt as a foreign body, like some dust particle in the eye, and annoying. If the mucus is plentiful and sticky, it is hard to blow out. You must loosen it with warm water or hose it with the rinse bottle. The sticky crusts in the nose block breathing during sleep, and in the morning, you wake up with a parched mouth and maybe a lump of sputum in the throat.

Most of these problems can be overcome with planning. The primary goal is hygiene. Get up in the morning, thoroughly rinse the nose, and try to get rid of any overnight crusts. Put on a face mask to reduce exposure to triggers of nasal secretions. Use the FEMA 4557 nebulizer or the rinsing solution. The nebulizer is very effective, but setting up the device can be cumbersome. On the other hand, the rinse bottle is something you can put in your pocket and use on an impromptu basis. Repeat these procedures as needed. Try to get a deluxe nose hair trimmer. That helps, too. My wife says I am no longer snoring as much. When you understand the dominant, overall treatment strategy as getting rid of excess mucus, you know what to do, and the discomforts of rhinitis will be history.”

In summary, the drug target, the TRPM8 receptor on nerve fibers of the OMC, can be reached with three delivery methods with optimal efficacy (Table 8). For severe rhinitis, an electronic compressor-nebulizer may offer the best control and focus of the delivered dose. The rinse bottle is an attractive alternative for general use because of its low expense, ease of use, and familiarity. The patient-reported outcomes to FEMA 4557 favor increased nasal patency, comfortable breathing, and there is also an objective index of decreased output of nasal exudates measured with a microbalance. These results offer a new strategy for the treatment of rhinitis and sinusitis.

TABLE 8
Methods for intransal delivery, and estimated dose (volxconc.)*

| Delivery Method | Formulation | mg/mL | mg |
|---|---|---|---|
| Compressor nebulizer | 1% ethanol, 2% polysorbate 80, | ~10-15 | ≥5 |
| (NasoNeb) | isotonic saline | | |
| Nose drops | Same as above | ~10 | ~1-2 |
| Rinse/irrigation bottle | 1% ethanol, saline | ~1 | ~1-2 |

*The rinse method estimated dose based on the retained volume of liquid in nose.

### REFERENCES

1. Erman, M. B. New development in physiological cooling agents. Perfumer and Flavorist Vol. 32, 2007.
2. Johnson S et al. Trigeminal Receptor Study of High-Intensity Cooling Agents. J Agric Food Chem. 2018; 66 (10): 2319-23.
3. Leffingwell J, Rowsell D. Wilkinson Sword cooling compounds: from the beginning to now. Perfume & Flavorist 39:34-44, 2014.
4. Leffingwell, J. C. Cooling ingredients and their mechanism of action. Handbook of cosmetic science and technology. Chpt. 65. Pp. 661-675. Barel, Paye, and Maibach. (edited). Informa Health Care, New York, 2009.
5. McKemy D D, Neuhausser W M, Julius D. Identification of a cold receptor reveals a general role for TRP channels in thermosensation. Nature. 2002; 416 (6876): 52-8.
6. Roswell D G et al. Acyclic carboxamides having a physiological cooling effect. US
7. Sherkheli M A et al. Characterization of Selective TRPM8 Ligands and their Structure. 2010; 13 (2): 242-53.
8. Smith R L et al. GRAS flavoring substances 24. Food Technol. 2009; 63 (6): 46-62.
9. Wei E T. Sensory/Cooling Agents for Skin Discomfort. 2012; 14 (2).
10. Wei, ET. 1,2,3,6-Tetrahydropyrimidine-2-one compositions and therapeutic methods therewith for pain and inflammation. U.S. Pat. No. 6,743,801. Jun. 1, 2004.
11. Wei, ET. [((1R,2S,5R)-2-Isopropyl-5-methyl-cyclohexanecarbonyl)-amino]-acetic acid isopropyl ester and related compounds and their use in therapy. U.S. Pat. No. 8,426,463. Apr. 23, 2013.
12. Wei E. T. and Thomas H. A. Anti-Inflammatory Peptide Agonists. Annual Review of Pharmacology and Toxicology 33:91-108, 1993.
13. Widdicombe J H, Wine J J. Airway gland structure and function. Physiol Rev. 2015; 95 (4): 1241-319.
14. Xu et al. Molecular mechanisms underlying menthol binding and activation of TRPM8 ion channel. Nat Commun. 2020. http://dx.doi.org/10.1038/s41467-020-17582-x)

1. A medication for the topical treatment of inflammation in the nasal cavity or paranasal sinuses of a subject in need of treatment thereof, comprising:
a liquid or semi-liquid medication having a therapeutically effective amount of N-ethyl-2,2-diisopropylbutanamide (Formula 1).

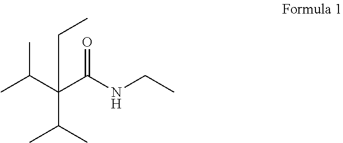

2. The medication as in claim 1 wherein the therapeutically effective amount is from about 1 to 20 mg per dose.

3. The medication as in claim 1 wherein the medication has from about 0.1% to 2% by weight of the Formula 1 compound.

4. The medication as in claim 1 wherein the inflammation being treated is rhinitis or rhinosinusitis.

5. The medication as in claim 1 wherein the inflammation being treated are the symptoms of nasal stuffiness, congestion, or obstructed breathing.

6. The medication as in claim 1 wherein the treatment of inflammation is treatment of mucus hypersecretion.

7. The medication as in claim 1 wherein the inflammation is caused by allergic and non-allergic rhinitis.

8. The medication as in claim 1 wherein the nasal inflammation is caused by allergy or allergies.

9. The medication as in claim 1 wherein the inflammation being treated are the symptoms of rhinitis or rhinosinusitis.

10. The medication as in claim 1 wherein the inflammation being treated are the symptoms of excess phlegm production.

11. The medication as in claim 10 wherein the treatment is the treatment of cough caused by excess phlegm in the pharynx.

12. The medication as in claim 1 wherein the medication is stored in and dispensed from a nasal irrigation system.

13. A pharmacological medication for the topical treatment of inflammation in a mammal in need of treatment thereof, comprising:
a liquid or semi-liquid medication having a therapeutically effective amount of a compound of Formula 2, wherein 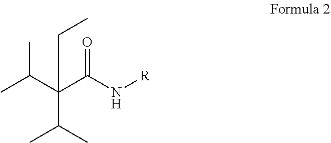
wherein R is n-propyl, cyclopropyl, or acetyl.

14. The medication as in claim 13 wherein the Formula 2 compound is N-(ethyl)-2,2-diisopropylbutanamide.

15. The medication as in claim 13 wherein the Formula 2 compound is N-(isopropyl)-2,2-diisopropylbutanamide.

16. The medication as in claim 13 wherein the Formula 2 compound is N-(cyclopropyl)-2,2-diisopropylbutanamide.

17. The medication as in claim 13 wherein the Formula 2 compounds is N-(acetyl)-2,2-diisopropylbutanamide.

18. The medication as in claim 13 wherein the medication has from about 0.1% to 2% by weight of the Formula 2 compounds.

19. The medication as in claim 13 wherein the medication is stored in and dispensed from a nasal irrigation system.

20. An apparatus for the topical treatment of inflammation in the nasal cavity or paranasal sinuses of a subject in need of treatment thereof, comprising:
a nasal irrigator system; and
a liquid or semi-liquid medication having a therapeutically effective amount of N-ethyl-2,2-diisopropylbutanamide, suspended or dissolved in an ethanol and saline solution and adapted to be administered by the nasal irrigator system to the subject.

21. The apparatus as in claim 20 wherein the nasal irrigator system is an electronically controlled nebulizer.

22. The apparatus as in claim 20 wherein the therapeutically effective amount is from about 1 to 20 mg per dose.

23. The apparatus as in claim 20 wherein the nasal irrigator system includes a bottle adapted to contain about 60 to 300 mL of liquid, the bottle being of sufficient construction to generate a plume after manual compression of the bottle.
